(19) United States
(12) Patent Application Publication
(10) Pub. No.: US 2008/0125794 A1
(43) Pub. Date: May 29, 2008

(54) SURGICAL INSTRUMENT

(75) Inventors: David L. Brock, Natick, MA (US); Woojin Lee, Hopkinton, MA (US)

Correspondence Address: Vista IP Law Group LLP, 2040 MAIN STREET, 9TH FLOOR, IRVINE, CA, 92614, US

(73) Assignee: HANSEN MEDICAL, INC., Mountain View, CA (US)

(21) Appl. No.: 12/024,094

(22) Filed: Jan. 31, 2008

Related U.S. Application Data
(63) Continuation of application No. 11/562,960, filed on Nov. 22, 2006.
Application No. 11/562,960 is a continuation of application No. 09/746,853, filed on Dec. 21, 2000, now Pat. No. 6,692,485.
Application No. 09/746,853 is a division of application No. 09/375,666, filed on Aug. 17, 1999, now Pat. No. 6,197,017.
Application No. 09/375,666 is a continuation of application No. 09/028,550, filed on Feb. 24, 1998, now abandoned.
Application No. 09/028,550 is a continuation-in-part of application No. 09/783,637, filed on Feb. 14, 2001, now abandoned.
Application No. 09/783,637 is a continuation of application No. PCT/US00/12553, filed on May 9, 2000.
Application No. PCT/US00/12553 is a continuation-in-part of application No. PCT/US01/11376, filed on Apr. 6, 2001.
Application No. PCT/US01/11376 is a continuation-in-part of application No. 09/746,853, filed on Dec. 21, 2000, now Pat. No. 6,692,485.
Application No. 09/746,853 is a continuation-in-part of application No. 09/827,503, filed on Apr. 6, 2001, now Pat. No. 6,432,112.
Application No. 09/827,503 is a continuation-in-part of application No. 09/746,853, filed on Dec. 21, 2000, now Pat. No. 6,692,485.
Application No. 09/746,853 is a continuation-in-part of application No. 09/827,503, filed on Apr. 6, 2001, now Pat. No. 6,432,112.
Application No. 09/827,503 is a continuation-in-part of application No. 09/827,643, filed on Apr. 6, 2001, now Pat. No. 6,554,844.
Application No. 09/827,643 is a continuation-in-part of application No. PCT/US00/12553, filed on May 9, 2000.

(51) Int. Cl. A61B 19/00

(52) U.S. Cl. 606/130

(57) ABSTRACT

A medical system comprises a rigid support affixed relative to a patient table, a carriage assembly mounted to the rigid support and a surgical instrument mounted to the carriage assembly, and a drive unit configured for pivoting the carriage assembly about the rigid support and for linearly moving the surgical instrument relative to the rigid support. The drive unit may be coupled to the surgical instrument via external cabling. Another medical system further comprises a rigid support affixed relative to a patient table, an adapter supported by the rigid support, an instrument insert removably disposed within the adapter, the instrument insert carrying a distal tool configured for performing a medical procedure on patient, and a drive unit configured for pivoting the adapter about the rigid support, linearly moving the adapter relative to the rigid support, and rotating the adapter about its longitudinal axis.

### CROSS REFERENCE TO RELATED APPLICATIONS

This application is a continuation of co-pending U.S. application Ser. No. 11/562,960, filed Nov. 22, 2006, which is a continuation of U.S. application Ser. No. 10/012,845, filed Nov. 16, 2001 (now U.S. Pat. No. 7,169,141), which is a continuation-in-part of and claims the benefit of priority from U.S. application Ser. No. 09/827,503, filed Apr. 6, 2001 (now U.S. Pat. No. 6,432,112), which is a continuation of U.S. application Ser. No. 09/746,853, filed Dec. 21, 2000 (now U.S. Pat. No. 6,692,485), which is a divisional of U.S. application Ser. No. 09/375,666, filed Aug. 17, 1999 (now U.S. Pat. No. 6,197,017), which is a continuation of U.S. application Ser. No. 09/028,550, filed Feb. 24, 1998 (now abandoned). This application is also a continuation-in-part of and claims the benefit of priority from U.S. application Ser. No. 09/783,637, filed Feb. 14, 2001 (now abandoned), which is a continuation of PCT Application Ser. No. PCT/US00/12553, filed May 9, 2000, which claims the benefit of priority of U.S. Provisional Application Ser. No. 60/133,407, filed May 10, 1999. This application is also a continuation-in-part of and claims the benefit of priority from PCT Application PCT/US01/11376, filed Apr. 6, 2001, which claims priority to U.S. application Ser. No. 09/746,853, filed Dec. 21, 2000 (now U.S. Pat. No. 6,692,485), and Ser. No. 09/827,503, filed Apr. 6, 2001 (now U.S. Pat. No. 6,432,112). This application is also a continuation-in-part of and claims the benefit of priority from U.S. application Ser. No. 09/746,853, filed Dec. 21, 2000 (now U.S. Pat. No. 6,692,485) and Ser. No. 09/827,503, filed Apr. 6, 2001 (now U.S. Pat. No. 6,432,112). This application is also a continuation-in-part of and claims the benefit of priority from U.S. application Ser. No. 09/827,643, filed Apr. 6, 2001 (now U.S. Pat. No. 6,554,844), which claims priority to, inter alia, U.S. Provisional Application Ser. No. 60/257,869, filed Dec. 21, 2000 and U.S. Provisional Application Ser. No. 60/195,264, filed Apr. 7, 2000, and is also a continuation-in-part of PCT Application PCT/US00/12553, filed May 9, 2000 from which U.S. application Ser. No. 09/783,637, filed Feb. 14, 2001 (now abandoned), claims priority.

This application also claims the benefit of priority under 35 U.S.C. §§ 119 and 120 to U.S. Provisional Application Ser. No. 60/293,346, filed May 24, 2001, U.S. Provisional Application Ser. No. 60/279,087, filed Mar. 27, 2001, U.S. Provisional Application Ser. No. 60/313,496, filed Aug. 21, 2001, U.S. Provisional Application Ser. No. 60/313,497, filed Aug. 21, 2001, U.S. Provisional Application Ser. No. 60/313,495, filed Aug. 21, 2001, U.S. Provisional Application Ser. No. 60/269,203, filed Feb. 15, 2001, U.S. Provisional Application Ser. No. 60/269,200, filed Feb. 15, 2001, U.S. Provisional Application Ser. No. 60/276,151, filed Mar. 15, 2001, U.S. Provisional Application Ser. No. 60/276,217, filed Mar. 15, 2001, U.S. Provisional Application Ser. No. 60/276,086, filed Mar. 15, 2001, U.S. Provisional Application Ser. No. 60/276,152, filed Mar. 15, 2001, U.S. Provisional Application Ser. No. 60/257,816, filed Dec. 21, 2000, U.S. Provisional Application Ser. No. 60/257,868, filed Dec. 21, 2000, U.S. Provisional Application Ser. No. 60/257,867, filed Dec. 21, 2000, and U.S. Provisional Application Ser. No. 60/257,869, filed Dec. 21, 2000.

The disclosures of all of the foregoing applications and U.S. Pat. No. 6,197,017 are all incorporated herein by reference in their entirety.

This application is also related to application Ser. Nos. ______ (Attorney Docket No. HNMD-EA004 CON2), ______ (Attorney Docket No. HNMD-EA004 CON3), ______ (Attorney Docket No. HNMD-EA004 CON4) and ______ (Attorney Docket No. HNMD-EA004 CON5), all of which are filed on the same date herewith.

The disclosures of the foregoing applications are expressly incorporated herein by reference. This application further expressly incorporates herein by reference, U.S. application Ser. Nos. 10/014,145 (now U.S. Pat. No. 6,775,582), 10/012,845 (now U.S. Pat. No. 7,169,141), 10/008,964 (now abandoned), 10/013/046 (now abandoned), 10/011,450 (now abandoned), 10/008,457 (now U.S. Pat. No. 6,949,106), 10/008,871 (now U.S. Pat. No. 6,843,793), 10/023,024 (now abandoned), 10/011,371 (now U.S. Pat. No. 7,090,683), 10/011,449 (now abandoned), 10/010,150 (now U.S. Pat. No. 7,214,230), 10/022,038 (now abandoned), 10/012,586, all filed on Nov. 16, 2001.

### BACKGROUND OF THE INVENTION

The present invention relates to surgical instruments and more particularly to surgical instruments which are remotely controlled by electronic control signals generated by a user which are sent to a drive unit which drives mechanically drivable components of a mechanical apparatus which support a surgical instrument.

### SUMMARY OF THE INVENTION

In accordance with a first aspect of the present inventions, the medical system comprises a rigid support affixed relative to a patient table. In one embodiment, the rigid support is a rigid post mounted to the patient table. The medical system further comprises a carriage assembly mounted to the rigid support and a surgical instrument mounted to the carriage assembly. The surgical instrument may a distal tool configured for performing a medical procedure on a patient. In one embodiment, the carriage assembly comprises at least one rail and a carriage slidably disposed on the rail(s), and the surgical instrument is affixed to the carriage. In another embodiment, the medical system further comprises a yoke mounted to the rigid support, and the carriage assembly further comprises a pivot piece pivotably secured to the yoke. The medical system may further comprise a bracket mounted to the rigid support, and a pin removably mounting the yoke to the rigid support

The medical system further comprises a drive unit (e.g., one having a motor array) configured for pivoting the carriage assembly about the rigid support and for linearly moving the surgical instrument relative to the rigid support. The drive unit may be coupled to the surgical instrument via external cabling. In one embodiment, the drive unit is further configured for rotating at least a portion of the surgical instrument about its longitudinal axis. In this case, the medical system may further comprise a base piece mounted to the carriage, and the drive unit may be configured for rotating the portion(s) of the surgical instrument within the base piece. In another embodiment, the drive unit is further configured for actuating the distal tool. In this case, the surgical instrument may comprise an adapter to which the drive unit is coupled and an instrument insert carrying the distal tool and removably disposed within the adapter, and the drive unit may be configured for actuating the distal tool through the adapter

In one embodiment, the medical system further comprises a remote controller configured for directing the drive unit to control movements of the surgical instrument. The remote controller may be coupled to the drive unit via external cabling, and may have a user interface for receiving commands from a user, which may be movements made at the user interface that correspond to movements of the surgical instrument.

In accordance with a second aspect of the present inventions, a medical system comprises a rigid support affixed relative to a patient table. In one embodiment, the rigid support is a rigid post mounted to the patient table. The medical system further comprises an adapter supported by the rigid support, and an instrument insert removably disposed within the adapter. The instrument insert carries a distal tool configured for performing a medical procedure on patient. In one embodiment, the adapter has an elongated guide member that receives the instrument insert.

The medical system further comprises a drive unit (e.g., one having a motor array) configured for pivoting the adapter about the rigid support, linearly moving the adapter relative to the rigid support, and rotating the adapter about its longitudinal axis. In one embodiment, the drive unit is further configured actuating the distal tool. The drive unit may be coupled to the surgical instrument via external cabling.

In another embodiment, medical system further comprises a remote controller configured for directing the drive unit to control movements of the surgical instrument. The remote controller may be coupled to the drive unit via external cabling, and may have a user interface for receiving commands from a user, which may be movements made at the user interface that correspond to movements of the surgical instrument.

### BRIEF DESCRIPTION OF THE DRAWINGS

FIG. 1 is a perspective view illustrating one embodiment of the robotic system of the present invention;

FIGS. 1A-1C are three views of a flexible cannula for use with the embodiment of FIG. 1;

FIG. 2A is a schematic diagram illustrating the degrees-of-freedom associated with the master station;

FIG. 2B is a schematic diagram illustrating the degrees-of-freedom associated with the slave station;

FIG. 2C shows a functional schematic diagram of the surgical adapter component of the system of FIG. 1;

FIG. 2D shows a functional schematic diagram of the instrument insert component of the system of FIG. 1;

FIG. 3 is a perspective view of the positioner assembly at the master station;

FIG. 4 is an exploded perspective view also of the positioner assembly at the master station;

FIG. 5 is a partially exploded view of the hand assembly portion associated with the positioner assembly;

FIG. 6 is a cross-sectional view of the hand assembly as taken along line 6-6 of FIG. 3;

FIG. 7 is a cross-sectional view at the master station as taken along lines 7-7 of FIG. 3;

FIG. 7A is a schematic perspective view of the yoke assembly portion of the positioner assembly;

FIG. 8 is a perspective view of the slave station;

FIG. 8A is a perspective view of an alternative adjustable clamp member at the slave station;

FIG. 8B is a top plan view of the clamp of FIG. 8A;

FIG. 8C is a side view of the clamp of FIGS. 8A and 8B as taken along line 8C-8C of FIG. 8B;

FIG. 8D is a perspective view of a template used with this embodiment;

FIG. 8E is a schematic cabling diagram illustrating one cable arrangement used to operate a tool;

FIG. 8F is an exploded perspective view of another version of the cable drive mechanism and tool in accordance with the present invention;

FIG. 8G is a schematic perspective view similar to that illustrated in FIG. 8F but specifically showing the cabling construction;

FIG. 8H is a partially broken away front elevational view as taken along line 8H-8H of FIG. 8F;

FIG. 8I is a top plan cross-sectional view taken along line 8I-8I of FIG. 8H;

FIG. 8J is a further cross-sectional top plan view as taken along line 8J-8J of FIG. 8H, FIG. 8K is a cross-sectional side view as taken along line 8K-8K of FIG. 8H;

FIG. 8L is a cross-sectional rear view of the coupler spindle and disk as taken along line 8L-8L of FIG. 8K.

FIG. 9 is a view at the slave station taken along line 9-9 of FIG. 8;

FIG. 10 is a side elevation view at the slave station taken along line 10-10 of FIG. 9;

FIG. 11 is a perspective view at the slave station;

FIG. 11A is a cross-sectional view as taken along line 11A-11A of FIG. 11;

FIG. 11B is a cross-sectional view as taken along line 11B-11B of FIG. 11A;

FIG. 11C is a cross-sectional view as taken along line 11C-11C of FIG. 11A;

FIG. 12 is a cross-sectional view as taken along line 12-12 of FIG. 11;

FIG. 13 is a cross-sectional view as taken along line 13-13 of FIG. 12;

FIG. 14 is a cross-sectional view as taken along line 14-14 of FIG. 12;

FIG. 15 is a perspective view at the slave station showing the instrument insert being removed from the adapter;

FIG. 15A is a top plan view of the instrument insert itself;

FIG. 16A is a perspective view at the tool as viewed along line 16A-16A of FIG. 11;

FIG. 16B is an exploded perspective view of the tool of FIG. 16A;

FIG. 16C is a fragmentary perspective view of an alternative tool referred to as a needle driver;

FIG. 16D is a side elevation view of the needle driver of FIG. 16C;

FIG. 16E is a perspective view of an alternate embodiment of the tool and wrist construction;

FIG. 16F is an exploded perspective view of the construction illustrated in FIG. 16E;

FIG. 16G is a fragmentary perspective view showing a portion of the bending section;

FIG. 16H is a plan view of the flexible wrist member associated with the construction of FIGS. 16E-16G.

FIG. 16I is a perspective view of still another embodiment of a flexible end tool;

FIG. 16J is an exploded perspective view of the construction illustrated in FIG. 16I;

FIG. 16K is a fragmentary perspective view showing further details of the bending section;

FIG. 17 is a perspective view of the drive unit at the slave station;

FIG. 17A is a schematic front view of the drive unit at the slave station;

FIG. 18 is a schematic perspective view of an alternative hand piece for use at the master station;

FIGS. 19A-19D are schematic diagrams showing alternate positions of the guide tube of the adapter;

FIG. 20 is a block diagram of the controller used with the robotic system of this embodiment;

FIG. 21 is a block diagram of further details of the controller, including the module board;

FIG. 22 is a block diagram of a control algorithm in accordance with the present embodiment; and

FIGS. 23-28 are a series of schematic diagrams of the input device position and resulting instrument position relating to the algorithm control of the present embodiment.

### DETAILED DESCRIPTION OF THE INVENTION

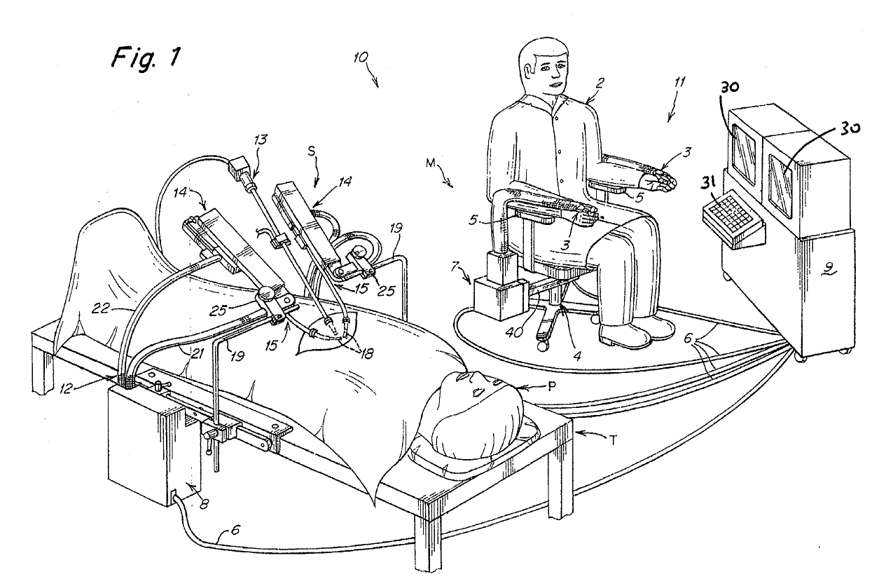

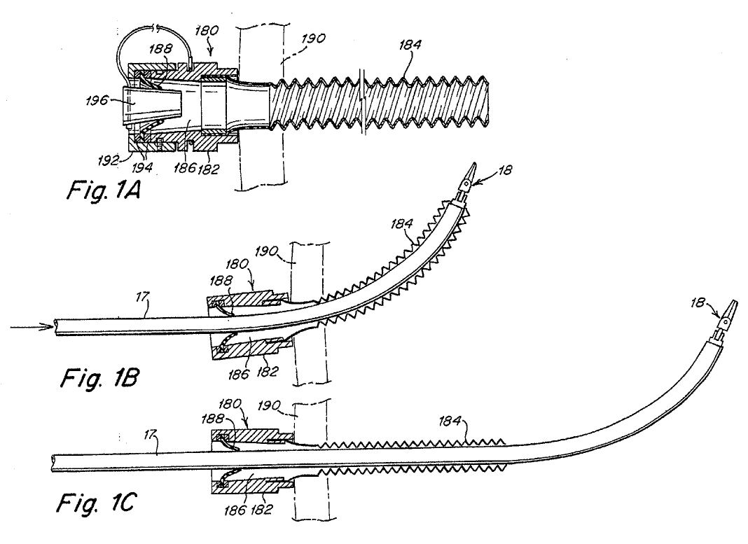

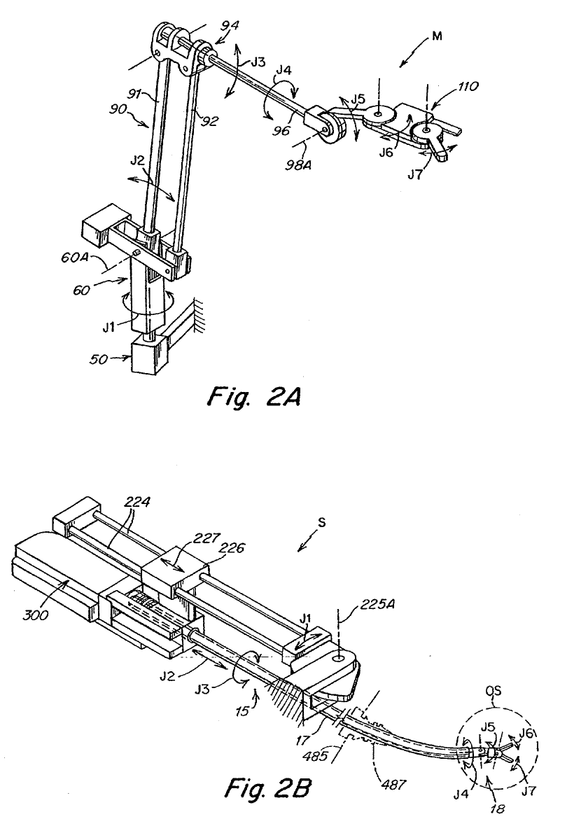

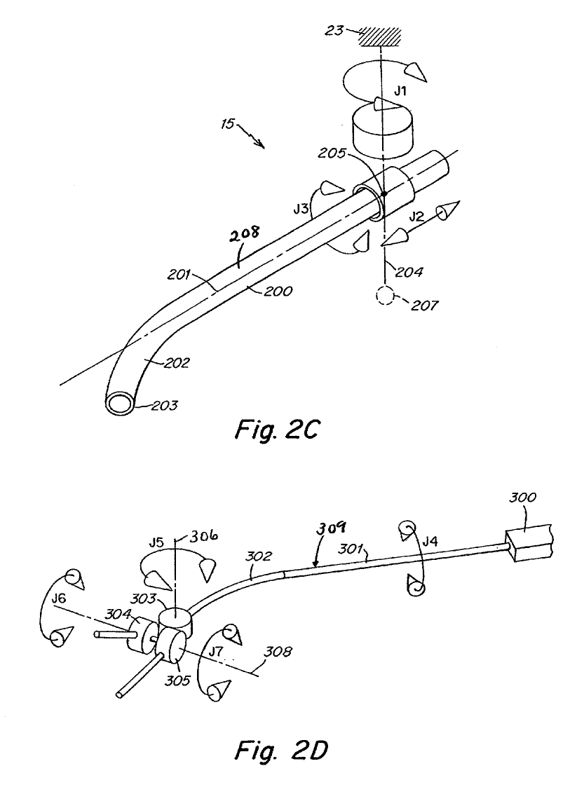

### A. Overview of Surgical Robotic System (FIGS. 1-2).

An embodiment of a surgical robotic system of the present invention is illustrated in the accompanying drawings. The described embodiment is preferably used to perform minimally invasive surgery, but may also be used for other procedures such as endoscopic or open surgical procedures.

FIG. 1. illustrates a surgical instrument system 10 that includes a master station M at which a surgeon 2 manipulates a pair of input devices 3, and a slave station S at which is disposed a pair of surgical instruments 14. The surgeon is seated in a comfortable chair 4 with his forearms resting upon armrests 5. His hands manipulate the input devices 3 which cause a responsive movement of the surgical instruments 14.

A master assembly 7 is associated with the master station M and a slave assembly 8 is associated with the slave station S. Assemblies 7 and 8 are interconnected by cabling 6 to a controller 9. Controller 9 has one or more display screens enabling the surgeon to view a target operative site, at which is disposed a pair of tools 18. The controller further includes a keyboard for inputting commands or data.

As shown in FIG. 1, the slave assembly 8, also referred to as a drive unit, is remote from the operative site and is positioned outside of the sterile field. In this embodiment, the sterile field is defined above the plane of the top surface of the operating table T, on which is placed the patient P. The drive unit 8 is controlled by a computer system, part of the controller 9. The master station M may also be referred to as a user interface, whereby commands issued at the user interface are translated by the computer into an electrical signal received by drive unit 8. Each surgical instrument 14, which is tethered to the drive unit 8 through mechanical cabling, produces a desired responsive motion.

Thus, the controller 9 couples the master station M and the slave station S and is operated in accordance with a computer program or algorithm, described in further detail later. The controller receives a command from the input device 3 and controls the movement of the surgical instrument in a manner responsive to the input manipulation.

With further reference to FIG. 1, associated with the patient P are two separate surgical instruments 14, one on either side of an endoscope 13. The endoscope includes a camera mounted on its distal end to remotely view the operative site. The dashed line circle in FIG. 2B, labeled OS, is an example of the operative site). A second camera may be positioned away from the site to provide an additional perspective on the medical procedure or surgical operation. It may be desirable to provide the endoscope through an orifice or incision other than the one used by the surgical instrument. Here three separate incisions are shown, two for the surgical instruments 14, 14 and a centrally disposed incision for the viewing endoscope 13. A drape over the patient has a single opening for the three incisions.

Each of the two surgical instruments 14 is generally comprised of two basic components, an adaptor or guide member 15 and an instrument insert or member 16. The adaptor 15 is a mechanical device, driven by an attached cable array from drive unit 8. The insert 16 extends through the adaptor 15 and carries at its distal end the surgical tool 18. Detailed descriptions of the adapter and insert are found in later drawings.

Although reference is made to “surgical instrument” it is contemplated that this invention also applies to other medical instruments, not necessarily for surgery. These would include, but are not limited to catheters and other diagnostic and therapeutic instruments and implements.

In FIG. 1 there is illustrated cabling 12 coupling the instrument 14 to the drive unit 8. The cabling 12 is readily attachable and detachable from the drive unit 8. The surgical adaptor 15, which supports the instrument at a fixed reference point is of relatively simple construction and may be designed for a particular surgical application such as abdominal, cardiac, spinal, arthroscopic, sinus, neural, etc. As indicated previously, the instrument insert 16 is couplable and decouplable to the adaptor 15, and provides a means for exchanging instrument inserts, with then attached tools. The tools may include, for example, forceps, scissors, needle drivers, electrocautery, etc.

Referring again to FIG. 1, the overall system 10 includes a surgeon's interface 11, computer system or controller 9, drive unit 8 and surgical instruments 14. Each surgical instrument 14 is comprised of an instrument insert 16 extending through adapter 15. During use, a surgeon manipulates the input device 3 at the surgeon's interface 11, which manipulation is interpreted by controller 9 to effect a desired motion of the tool 18 within the patient.

Each surgical instrument 14 is mounted on a separate rigid support post 19 which is illustrated in FIG. 1 as removably affixed to the side of the surgical table T. This mounting arrangement permits the instrument to remain fixed relative to the patient even if the table is repositioned. Although two instruments 14 are shown here, the invention can be practiced with more or with only a single surgical instrument.

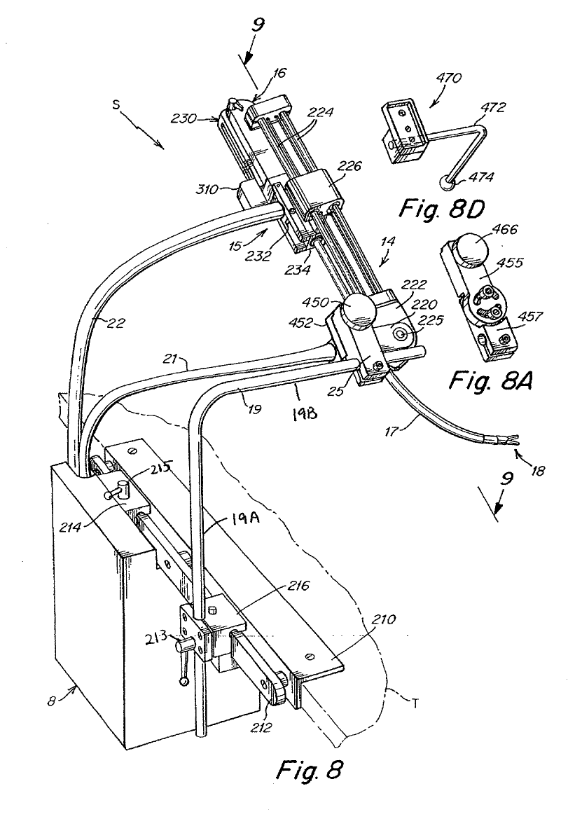

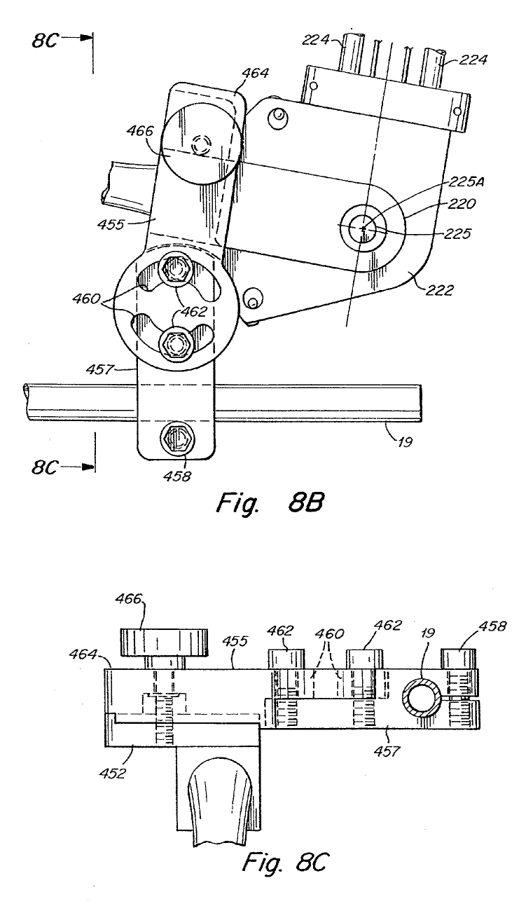

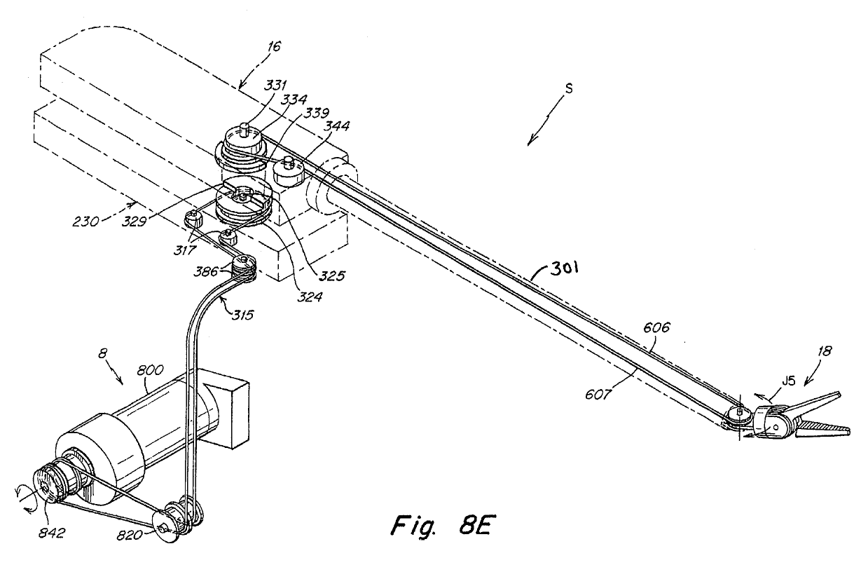

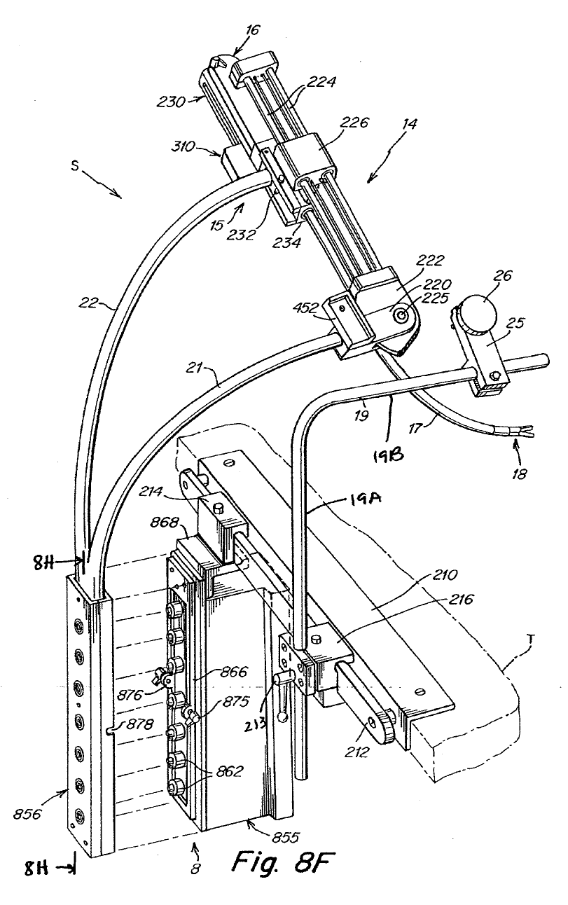

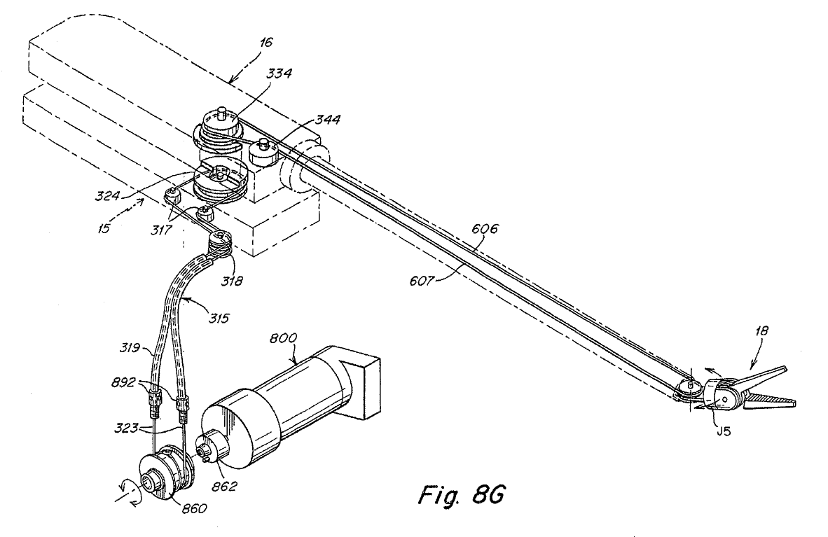

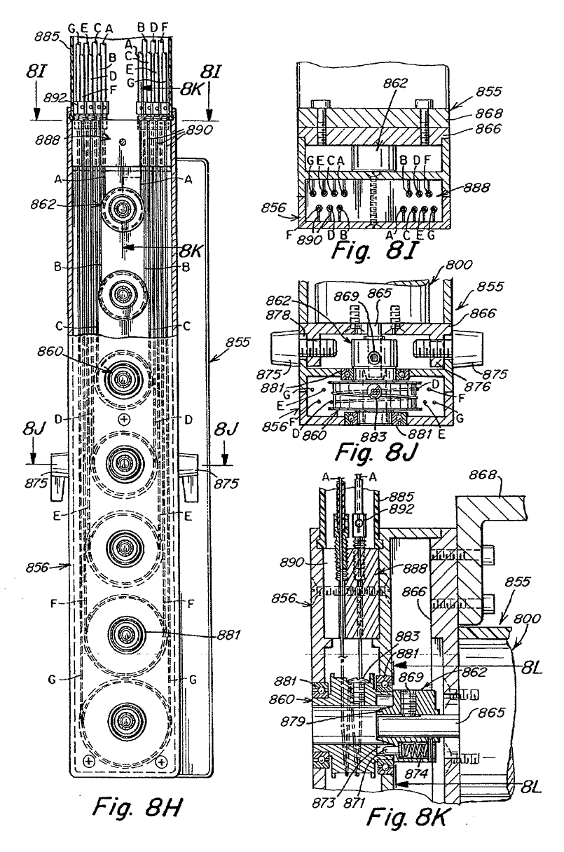

Each surgical instrument 14 is connected to the drive unit 8 by two mechanical cabling (cable-in-conduit) bundles 21 and 22. These bundles 21 and 22 terminate at connection modules, illustrated in FIG. 8F, which are removably attachable to the drive unit 8. Although two cable bundles are used here, more or fewer cable bundles may be used. Also, the drive unit 8 is preferably located outside the sterile field as shown here, although in other embodiments the drive unit may be draped with a sterile barrier so that it may be located within the sterile field.

In a preferred technique for setting up the system, a distal end of the surgical instrument 14 is manually inserted into the patient through an incision or opening. The instrument 14 is then mounted to the rigid post 19 using a mounting bracket 25. The cable bundles 21 and 22 are then passed away from the operative area to the drive unit 8. The connection modules of the cable bundles are then engaged to the drive unit 8. One or more instrument inserts 16 may then be passed through the surgical adaptor 15, while the adapter remains fixed in position at the operative site. The surgical instrument 14 provides a number of independent motions, or degrees-of-freedom, to the tool 18. These degrees-of-freedom are provided by both the surgical adaptor 15 and the instrument insert 16.

The surgeon's interface 11 is in electrical communication with the controller 9. This electrical control is primarily by way of the cabling 6 illustrated in FIG. 1 coupling from the master assembly 7. Cabling 6 also couples from the controller 9 to the drive unit 8. The cabling 6 is electrical cabling. The drive unit 8 however, is in mechanical communication with the instruments 14 in mechanical cabling 21, 22. The mechanical communication with the instrument allows the electromechanical components to be removed from the operative region, and preferably from the sterile field.

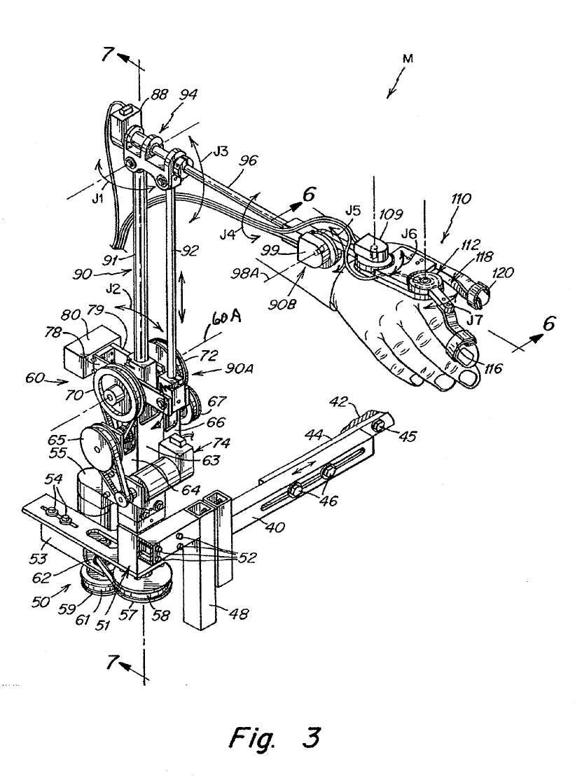

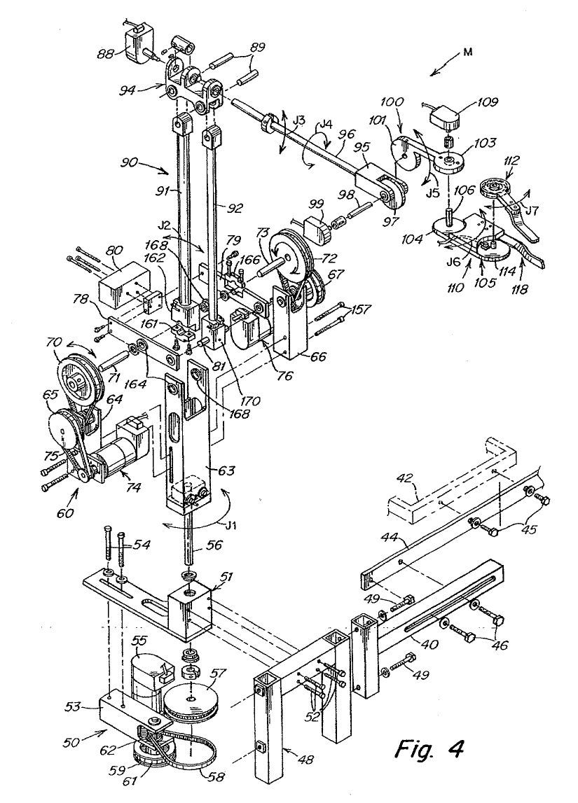

FIG. 2A illustrates the various movements (J1-J7) that occur at the master station M while FIG. 2B illustrates various movements (J1-J7) that occur at the slave station S. More specific details regarding FIGS. 2A and 2B are contained in a later discussion of FIGS. 3-4 (with regard to the master station of FIG. 2A) and FIGS. 8-9 (with regard to the slave station of FIG. 2B).

FIG. 2C is a simplified representation of adaptor 15 of the slave station, useful in illustrating the three degrees-of-freedom enabled by the adapter. The adapter as shown in FIG. 2C comprises a generally rigid outer guide tube 200 (corresponding to guide tube 17 in FIG. 2B) through which an inner flexible shaft, carrying a tool 18 at its distal end, is inserted into the patient. The adapter provides three degrees-of-freedom by way of a pivotal joint J1, a linear joint J2, and a rotary joint J3. From a fixed mounting point 23 shown schematically at the top of FIG. 2C, the pivotal joint J1 allows the guide tube 200 to pivot about a fixed vertical axis 204, while maintaining the tube (both the proximal straight portion 208 and distal curved portion 202) in a single plane, transverse to pivot axis 204, in which lies central horizontal tube axis 201. The linear joint J2, moves the rigid guide tube 200 along this same axis 201. The rotary joint J3 rotates the guide tube 200 about the tube axis 201. The guide tube 200 has a fixed curve or bend 202 at its distal end 203; as a result the distal end 203 will orbit in a circle about the axis 201 when the straight portion 208 of the guide tube 200 is rotated about its axis 201. Alternatively, the three degrees-of-freedom can be achieved by a structure other than a curve 202, such as by means of a joint or angular end section. The point is to have the distal end 203 of the tube 200 at a location spaced away from the tube axis 201.

FIG. 2C thus shows a schematic view of the three degrees of freedom of the rigid curved guide tube 200. In summary, via the pivot 205 the guide tube 200 may rotate in a direction J1 about an axis 204. The guide tube 200 may also slide in an axial direction J2 along proximal tube axis 201 (via the linear slider) and rotate in a direction J3 about the proximal tube axis 201 (via a rotatable mounting at the guide tube housing). It is intended that the point 205 at which the axes of linear movement and rotation 201 and 204 intersect, be in linear alignment (along axis 204) with the incision point illustrated in dotted outline at 207, at which the guide tube enters the patient. Positioning the incision 207 in substantially vertical linear alignment with point 205 results in less trauma to the patient in the area around the incision, because movement of the guide tube 17 near the point 205 is limited.

In addition to the three degrees-of-freedom provided by the guide tube 17, the tool 18 may have three additional degrees-of-freedom. This is illustrated schematically in FIG. 2D which shows an inner flexible shaft 309, fixed at its proximal end 300, having a straight proximal portion 301 and having a curved distal portion 302 with a tool 18 mounted at the distal end. The shaft 309 has a wrist joint that rotates about axis 306. A pair of pinchers 304, 305 independently rotate as shown (J6 and J7) about horizontal axis 308 to open and close (e.g., to grasp objects). Still further, the inner shaft can be rotated (J4) about the central axis of proximal portion 301.

In practice, an instrument insert 16 (carrying the inner shaft 309) is positioned within the adaptor 15 (including guide tube 17), so that the movements of the insert are added to those of the adaptor. The tool 18 at the distal end of insert 16 has two end grips 304 and 305, which are rotatably coupled to wrist link 303, by two rotary joints J6 and J7. The axis 308 of the joints J6 and J7 are essentially collinear. The wrist link 303 is coupled to a flexible inner shaft 302 through a rotary joint J5, whose axis 306 is essentially orthogonal to the axis 308 of joints J6 and J7. The inner shaft 309 may have portions of differing flexibility, with distal shaft portion 302 being more flexible than proximal shaft portion 301. The more rigid shaft portion 301 is rotatably coupled by joint J4 to the instrument insert base 300. The axis of joint J4 is essentially co-axial with the rigid shaft 301. Alternatively, the portions 301 and 302 may both be flexible.

Through the combination of movements J1-J3 shown in FIG. 2C, the adaptor 15 can position the curved distal end 203 of guide tube 200 to any desired position in three-dimensional space. By using only a single pivotal motion (J1), the motion of the adaptor 15 is limited to a single plane. Furthermore, the fixed pivot axis 204 and the longitudinal axis 201 intersect at a fixed point 205. At this fixed point 205, the lateral motion of the guide tube 200 is minimal, thus minimizing trauma to the patient at the aligned incision point 207.

The combination of joints J4-J7 shown in FIG. 2D allow the instrument insert 16 to be actuated with four degrees-of-freedom. When coupled to the adaptor 15, the insert and adaptor provide the instrument 14 with seven degrees-of-freedom. Although four degrees-of-freedom are described here for the insert 16, it is understood that greater and fewer numbers of degrees-of-freedom are possible with different instrument inserts. For example an energized insert with only one gripper may be useful for electro-surgery applications, while an insert with an additional linear motion may provide stapling capability.

FIG. 2B shows in dotted outline a cannula 487, through which the guide tube 17 is inserted at the incision point. Further details of the cannula are illustrated in FIGS. 1A-1C. FIG. 1A is a longitudinal cross-sectional view showing a cannula 180 in position relative to, for example, an abdominal wall 190 of the patient. FIG. 1B is a schematic view of the guide tube 17 being inserted through the flexible cannula 180. FIG. 1C is a schematic view of the guide tube inserted so that the proximal straight section of the tube is positioned at the incision point within the cannula, with the curved distal end of the guide tube and tool 18 disposed at a target or operative site.

The cannula 180 includes a rigid base 182 and a flexible end or stem 184. The base may be constructed of a rigid plastic or metal material, while the stem may be constructed of a flexible plastic material having a fluted effect as illustrated in FIGS. 1A-1C. The length of the base is short enough that the curve in the guide tube can easily pass through a center passage or bore 186 in the base 182. The bore 186 has a larger diameter than the outer diameter of the guide tube 17 to facilitate passage of the guide tube through the cannula 180. A diaphragm or valve 188 seals the guide tube 17 within the cannula 180.

FIG. 1A shows a cap 192 secured to the proximal end of the base 182 by one or more o-rings 194. Before the guide tube 17 is inserted in cannula 180, a plug 196 may be inserted to seal the proximal end of the base 182. The plug 196 is secured by a tether 198 to base 182.

In the context of an insertable instrument system, there may generally be distinguished two types of systems, flexible and rigid. A flexible system would use a flexible shaft, which may be defined as a shaft atraumatically insertable in a body orifice or vessel which is sufficiently pliable that it can follow the contours of the body orifice or vessel without causing significant damage to the orifice or vessel. The shaft may have transitions of stiffness along its length, either due to the inherent characteristics of the material comprising the shaft, or by providing controllable bending points along the shaft. For example, it may be desirable to induce a bend at some point along the length of the shaft to make it easier to negotiate a turn in the body orifice. A mechanical bending of the tube may be caused by providing one or more mechanically activatable elements along the shaft at the desired bending point, which a user remotely operates to induce the bending upon demand. The flexible tube may also be caused to bend by engagement with a body portion of greater stiffness, which may, for example, cause the tube to bend or loop around when it contacts the more stiffer body portion. Another way to introduce a bend in the flexible shaft is to provide a mechanical joint, such as the wrist joint provided adjacent to tool 18 as previously described, which, as discussed further, is mechanically actuated by mechanical cabling extending from a drive unit to the wrist joint.

One potential difficulty with flexible shafts or tubes as just described is that it can be difficult to determine the location of any specific portion or the distal end of such shaft or tube within the patient. In contrast, what is referred to as a rigid system may utilize a rigid guide tube 17 as previously described, for which the position of the distal end is more easily determined, simply based upon knowing the relevant dimensions of the tube. Thus, in the system previously described, a fixed pivot point (205 in FIG. 2C) is aligned with an incision point 207. One can determine the position of the rigid guide tube 17, knowing the length from the fixed point to the distal end of the guide tube, which is fixed and predetermined based upon the rigid nature of the guide tube, and the known curvature of the distal end of the guide tube. The point of entry or incision point serves as a pivot point, for which rotation J1 of the guide tube about the fixed axes 204 is limited to maintaining the proximal end of the guide tube in a single plane.

Furthermore, by inserting the more flexible shaft, carrying a tool 18 at its distal end, within the rigid guide tube, the rigid guide tube in effect defines a location of the flexible shaft and its distal end location tool 18.

Also relevant to the present invention is the use of the term “telerobotic” instrument system, in which a physician or medical operator is manually manipulating some type of hand tool, such as a joy stick, and at the same time is looking at the effect of such manual manipulation on a tool which is shown on a display screen, such as a television or a video display screen, accessible to the operator. The operator then can adjust his manual movements in response to visual feedback he receives by viewing the resulting effect on the tool, shaft guide tube, or the like, shown on the display screen. It is understood that the translation of the doctor's manual movement, via a computer processor which feeds a drive unit for the inserted instrument, is not limited to a proportional movement, rather, the movement may be scaled by various amounts, either in a linear fashion or a nonlinear fashion. The scaling factor may depend on where the instrument is located or where a specific portion of the instrument is located, or upon the relative rate of movement by the operator. The computer controlled movement of the guide tube or insert shaft in accordance with the present invention, enables a higher precision or finer control over the movement of the instrument components within the patient.

In practice, the physician, surgeon or medical operator would make an incision point, inserting the flexible cannula previously shown. He would then manually insert the rigid curved guide tube until the distal point of the guide tube was positioned at the operative site. With the guide tube aligned in a single plane, the operator would clamp the guide tube at the support bracket 25 on post 19, to establish the fixed reference pivot point, (205 in FIG. 2C), with the incision point axially aligned under the fixed pivot point. The operator would then manually insert the instrument insert through the guide tube until the tool 18 is extended out from the distal end of the guide tube. The wrist joint on the inner insert shaft is then positioned at a known point, based upon the known length and curvature of the rigid guide tube and distance along that length at which the incision point is disposed. Then, a physician, surgeon or medical operator located at the master station can manually adjust the hand assembly to cause a responsive movement of the inserted instrument. The computer control decides what the responsive movement at the instrument is, including one or more of movement of the guide tube, the whole instrument 14, or the flexible inner shaft or the tool at its distal end. A pivotal movement J1 will rotate the proximal end of the guide tube, causing pivoting of the whole instrument 14. An axial movement J2 of the whole instrument 14 will reposition the instrument in the single plane. A rotational movement J3 of just the guide tube results in the end of the guide tube and end of the inner shaft being taken out of the plane, following a circular path or orbit in accordance with rotation of the guide tube shaft. These three movements J1, J2 and J3 are defined as setting the position of the wrist joint 303 of the tool.

The other three movements J4-J7, are defined as setting the orientation of the instrument insert, and more specifically, a direction at which the tool is disposed with respect to the wrist joint. Central mechanical cables in the inner shaft cause motions J5-J7, J5 being the wrist movement and J6-J7 being the jaw movement of the tool. The J4 movement is for rotation of the inner shaft by its proximal axis, within the guide tube. These relative movements, and the position and orientation of the instrument insert, will be further described in a later discussion of an example of the computer algorithm for translating the movement at the master station to a movement at the slave station.

### B. The Master Station M (FIGS. 3-7)

At the master station illustrated FIG. 1 and shown in further detail in FIG. 3, there are two sets of identical hand controls, one associated with each hand of the surgeon. The outputs of both controls are fed to assembly 7, which is secured to the surgeon's chair 4 by a cross-brace 40. In FIG. 3, the brace 40 is shown secured to the chair frame 42 by means of adaptor plate 44 and bolts 45. Additional bolts 46, with associated nuts and washers secure the cross-brace 40 in a desired lateral alignment (see double headed arrow) along the adaptor plate 44. Additional bolts 49 (see FIG. 4) are used for securing the cross-brace 40 with a base piece 48. The base piece 48 supports lower and upper positioner assemblies, as will now be described.

A lower positioner assembly 50 is supported from the base piece 48. An upper positioner assembly 60 is supported above and in rotational engagement (see arrow J1 in FIGS. 2A and 4), in a substantially horizontal plane with the lower positioner assembly. This rotational movement J1 enables a lateral or side-to-side manipulation by the surgeon. An arm assembly 90, having a lower proximal end 90A, is pivotally supported (J2) from the upper positioner assembly 60 about a substantially horizontal axis 60A (see FIGS. 2A and 3) to enable substantially vertical surgeon manipulation. The arm assembly 90 has an upper distal end 90B (FIG. 3), carrying a hand assembly 110.

As shown in FIG. 4, the lower positioner assembly 50 includes a base member 51 that is secured to the base piece 48 by bolts 52. It also includes a bracket 53 that is secured to the base member 51 by means of bolts 54. The bracket 53 supports a motor/encoder 55. A vertical shaft 56 that extends from the upper positioner assembly 60 to the base member 51, extends through a passage in the base member 51 and is secured to a pulley 57 disposed under the base member 51. A belt 58 engages with pulley 57 and with a further pulley 62 supported from the bracket 53. This further pulley 62 is on a shaft that engages a pulley 59. A further belt 61 intercouples pulley 59 to the shaft of the motor/encoder 55.

In FIGS. 3 and 4, the base member 51 and bracket 53 are stationary; however, upon rotation about J1, drive is applied to the pulleys 57 and 59 thus applying drive to the motor/encoder 55. This detects the position and movement from one position to another of the upper positioner assembly 60 relative to the lower positioner assembly 50.

The upper positioner assembly 60 has a main support bracket 63, supporting on either side thereof side support brackets 64 and 66. Side bracket 64 supports a pulley 65, while side bracket 66 supports a pulley 67. Above pulley 65 is another pulley 70, while above pulley 67 is another pulley 72. Pulley 70 is supported on shaft 71, while pulley 72 is supported on shaft 73.

Also supported from side support bracket 64 is another motor/encoder 74, disposed on one side of the main support bracket 63. On the other side of bracket 63 is another motor/encoder 76, supported from side support bracket 66. Motor/encoder 74 is coupled to the shaft 71 by pulleys 65 and 70 and associated belts, such as the belt 75 disposed about pulley 65. Similarly, motor/encoder 76 detects rotation of the shaft 73 through pulleys 67 and 72 by way of two other belts. The pulley 65 is also supported on a shaft coupling to pulley 70 supported by side support bracket 64. A further belt goes about pulley 70 so there is continuity of rotation from the shaft 71 to the motor/encoder 74. These various belts and pulleys provide a movement reduction ratio of, for example, 15 to 1. This is desirable so that any substantial movements at the master station are translated as only slight movements at the slave station, thereby providing a fine and controlled action by the surgeon.

Extending upwardly from main support bracket 63, is arm assembly 90 which includes a pair of substantially parallel and spaced apart upright proximal arms 91 and 92, forming two sides of a parallelogram. Arm 91 is the main vertical arm, while arm 92 is a tandem or secondary arm. The bottoms of arms 91 and 92 are captured between side plates 78 and 79. The secondary arm 92 is pivotally supported by pin 81 (see FIG. 4) from the forward end of the side plates 78 and 79. The main arm 91 is also pivotally supported between the side plates 78 and 79, but is adapted to rotate with the shaft 71. Thus, any forward and back pivoting J2 of the arm 91 is sensed through the shaft 71 down to the motor/encoder 74. This movement J2 in FIGS. 2A and 4 translates the forward and rearward motion at the surgeon's shoulder.

The side plates 78 and 79 pivot on an axis defined by shafts 71 and 73. However, the rotation of the plates 78 and 79 are coupled only to the shaft 73 so that pivotal rotation, in unison, of the side plates 78 and 79 is detected by motor/encoder 76. This action is schematically illustrated in FIGS. 2A and 4 by J3. Movement J3 represents an up and down motion of the surgeon's elbow. A counterweight 80 is secured to the more rear end of the side plates 78 and 79, to counter-balance the weight and force of the arm assembly 90.

As depicted in FIGS. 3 and 4, the tops of the arms 91 and 92 are pivotally supported in a bracket 94 by two pivot pins 89. The bracket 94 also supports a distal arm 96 of the arm assembly 90. The rotation of distal arm 96 is sensed by an encoder 88. Thus, the distal arm 96 is free to rotate J4 about its longitudinal axis, relative to the arms 91 and 92. This rotation J4 translates the rotation of the surgeon's forearm.

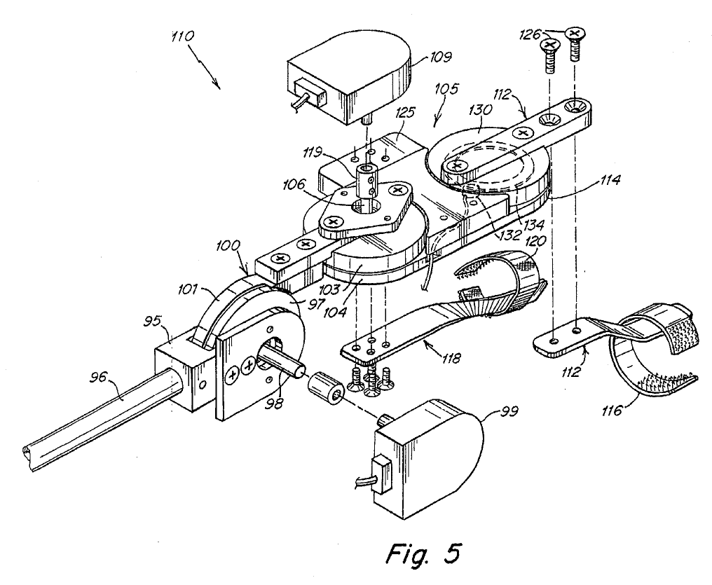

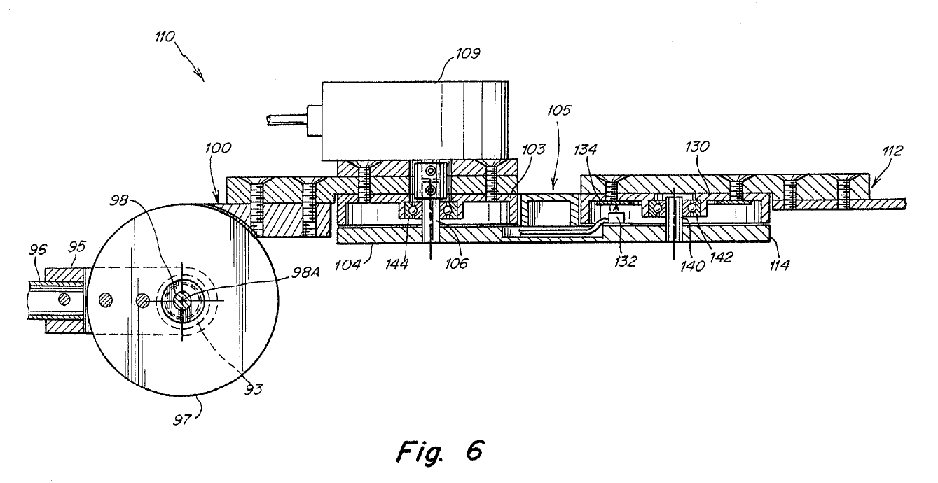

The distal end of distal arm 96 is forked, as indicated at 95 in FIG. 4. The forked end 95 supports disc 97 in a fixed position on shaft 98. The disc 97 is fixed in position while the shaft 98 rotates therein; bearings 93 support this rotation. The shaft 98 also supports one end of pivot member 100, which is part of hand assembly 110. The pivot member 100 has at its proximal end a disc 101 that is supported co-axially with the disc 97, but that rotates relative to the fixed disc 97 (see FIGS. 5 and 6). This rotation is sensed by an encoder 99 associated with shaft 98. The disc end 101 of the pivot member 100 defines the rotation J5 in FIG. 4, which translates the wrist action of the surgeon, particularly the up and down wrist action.

The pivot member 100 has at its other end a disc 103 that rotates co-axially with a disc end 104 of hand piece 105. There is relative rotation between disc 103 and disc 104 about a pivot pin 106 (see FIGS. 4 and 6). This relative rotation between the pivot member 100 and the hand piece 105 is detected by a further encoder 109 associated with discs 103 and 104. This action translates lateral or side to side (left and right) action of the surgeon's hand.

At the very distal end of the master station is a forefinger member 112 that rotates relative to end 114 of the hand piece 105. As indicated in FIG. 3, the forefinger piece 112 has a Velcro loop 116 for holding the surgeon's forefinger to the piece 112. Also extending from the hand piece 105 is a fixed position thumb piece 118, with an associated Velcro loop 120. In FIG. 3, motion J7 represents the opening and closing between the surgeon's forefinger and thumb.

Reference is now made to FIG. 5, which shows expanded details of the distal end of the arm assembly. One end of the distal arm 96 couples to the fork 95; fork 95 supports one end of the pivot member 100. The encoder 99 detects the position of the pivot member 100 relative to the distal arm 96. The encoder 109 couples to a shaft adapter 119 and detects relative displacement between the pivot member 100 and the hand piece 105. The thumb piece 118 is secured to the side piece 125 which, in turn, is secured as part of the hand piece 105. Bolts 126 secure the finger piece 112 to the rotating disc 130. The distal end encoder 132, with encoding disc 134, detects the relative movement between the surgeon's thumb and forefinger pieces.

FIG. 6 shows further details of the distal end of the arm assembly. Pivot member 100 is attached to the distal arm 96 and the hand piece 105. Further details are shown relating to the encoder 132 and the encoder disc 134. A shaft 140, intercoupling hand piece 105 and disc 130, is supported by a bearing 142. The shaft 106 is also supported by a bearing 144.

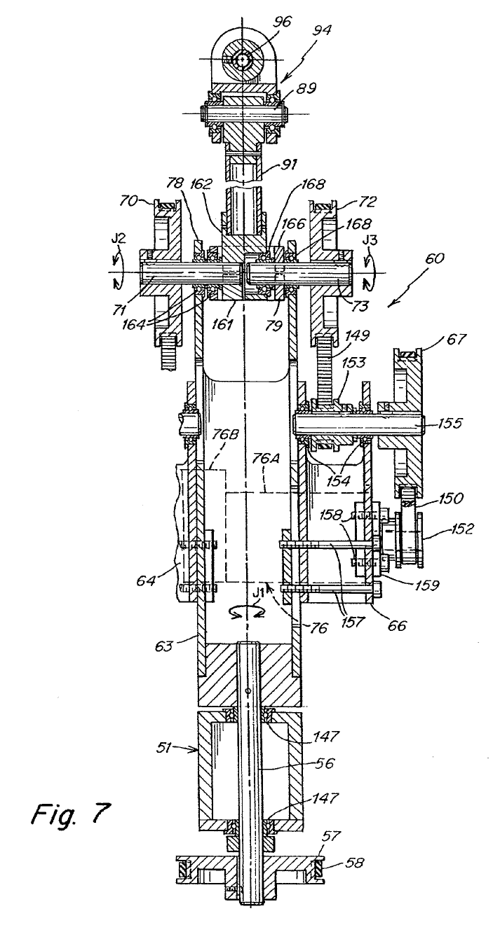

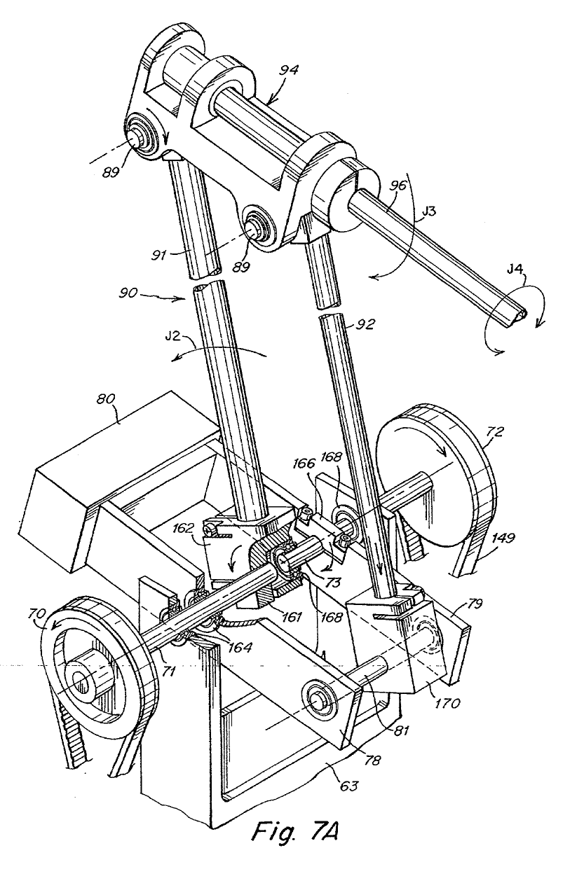

The detailed cross-sectional view of FIG. 7 is taken along lines 7-7 of FIG. 3. This illustrates the base member 51 with the pulley 57 supported thereunder by means of the shaft 56. Also illustrated are bearings 147 about shaft 56 which permit the main support bracket 63 to pivot (J1). Pulley 57 rotates therewith and its rotation is coupled to the encoder 55 for detecting the J1 rotation. FIG. 7 also illustrates the motor/encoder 76, where the separate dashed portions identify motor 76A and encoder 76B.

FIG. 7 also shows further details of the belt and pulley arrangement. For simplicity, only the pulley 67 and its associated support is disclosed. Substantially the same construction is used on the other side of the main support bracket 63 for the mounting of the opposite pulley 65. A belt 149 about pulley 72 also engages with pulley 153 fixedly supported on the shaft 155. The shaft 155 rotates relative to the fixed side support bracket 66, by way of bearings 154. The shaft 155 supports the pulley 67. A toothed belt 150 is disposed about pulley 67 to the smaller pulley 152. The pulley 152 is supported on the shaft of the motor/encoder 76. For the most part all pulleys and belts disclosed herein are toothed so that there is positive engagement and no slippage therebetween.

In order to provide adjustment of the belts 149 and 150, adjusting screws are provided. One set of adjusting screws is shown at 157 for adjusting the position of the side support bracket 66 and thus the belt 149. Also, there are belt adjusting screws 158 associated with support plate 159 for adjusting the position of the encoder and thus adjusting the belt 150.

FIG. 7 also illustrates the pulleys 70 and 72 with their respective support shafts 71 and 73. FIG. 7A shows details of the pulleys 70 and 72 and their support structure. The pulley 70 is associated with motion J2. The pulley 72 is associated with motion J3. The pulley 70 and its associated shaft 71 rotate with the vertical main shaft or arm 91. The pulley 72 and its associated shaft 73 rotate independent of the arm 91 and instead rotate with the rotation of the side plates 78 and 79. One end of shaft 71 is secured with the pulley 70. The other end of the shaft 71 engages a clamp 161, which clamps the other end of the shaft to the support piece 162 of the main vertical arm 91. The shaft 71 is supported for rotation relative to the main support bracket 63 and the side plates 78,79 by means of bearings 164.

The opposite pulley 72 and its shaft 73 are supported so that the pulley 72 rotates with rotation of the yoke formed by side plates 78 and 79. A clamp 166 clamps the shaft 73 to the side plate and thus to the rotating yoke. This yoke actually rotates with the pin of shaft 73. For further support of the shaft 73, there are also provided bearings 168, one associated with the support bracket 63 and another associated with support piece 162.

Regarding the yoke formed by side plates 78 and 79, at one end thereof is a counterweight 80, as illustrated in FIGS. 4 and 7A. The other end supports a rotating block 170 (see FIG. 4) that supports the lower end of arm 92 and has oppositely disposed ends of pin 81 rotatably engaged with that end of the yoke (side plates 78 and 79). Bushings or bearings may be provided to allow free rotation of the bottom end of the arm 92 in the yoke that captures this arm.

In practice, the following sequence of operations occur at master station M. After the instrument 14 has been placed at the proper operative site, the surgeon is seated at the console and presses an activation button, such as the “enter” button on the keyboard 31 on console 9. This causes the arms at the master station to move to a predetermined position where the surgeon can engage thumb and forefinger grips. FIG. 1 shows such an initial location where the arm assemblies 3 are essentially pointed forward. This automatic initialization movement is activated by the motors in unit 7 at the master station. This corresponds, in FIG. 2A, to upper arm 96 being essentially horizontal and lower arm 92 being essentially vertical.

While observing the position of the tools on the video display screen 30, the surgeon now positions his hand or hands where they appear to match the position of the respective tool 18 at the operative site (OS in FIG. 2B). Then, the surgeon may again hit the “enter” key. This establishes a reference location for both the slave instrument and the master controls. This reference location is discussed later with details of controller 9 and an algorithm for controlling the operations between the master and slave stations. This reference location is also essentially identified as a fixed position relative to the wrist joint at the distal end of distal arm 96 at pin 98 in FIG. 4 (axis 98A in FIG. 2A). This is the initial predefined configuration at the master station, definable with three dimensional coordinates.

Now when the surgeon is ready to carry out the procedure, a third keystroke occurs, which may also be a selection of the “enter” key. When that occurs the motors are activated in the drive unit 8 so that any further movement by the surgeon will initiate a corresponding movement at the slave end of the system.

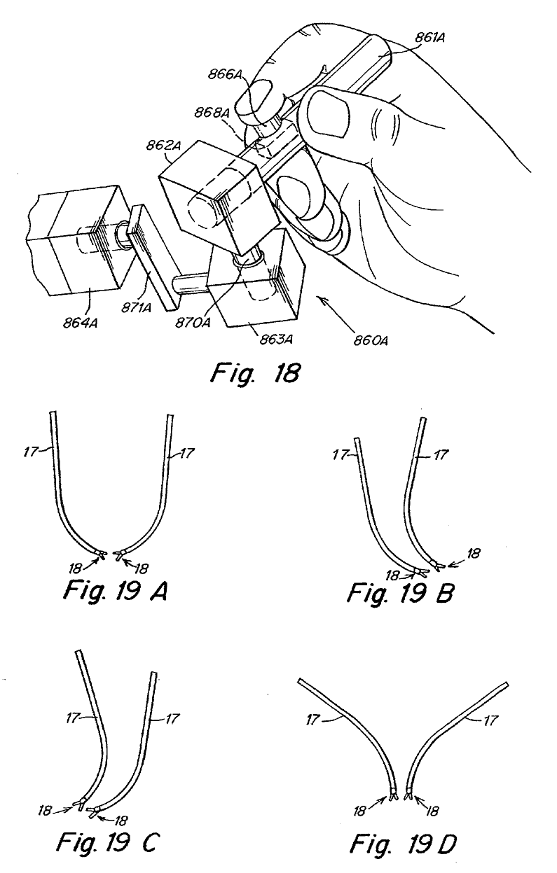

Reference is now made to FIG. 18 which is a schematic perspective view of an alternate embodiment of an input device or hand assembly 860A. Rather than providing separate thumb and forefinger members, as illustrated previously, the surgeon's hand is holding a guide shaft 861A. On the shaft 861 there is provided a push-button 866A that activates an encoder 868A. The guide shaft 861A may be considered more similar to an actual surgical instrument intended to be handled directly by the surgeon in performing unassisted nonrobotic surgery. Thus, the hand piece 860A illustrated in FIG. 18 may be more advantageous to use for some types of operative procedures.

FIG. 18 illustrates, in addition to the encoder 868A, three other encoder blocks, 862A, 863A and 864A. These are schematically illustrated as being intercoupled by joints 870A and 871A. All four of these encoders would provide the same joint movements depicted previously in connection with joints J4-J7. For example, the button 866A may be activated by the surgeon to open and close the jaws.

### C. The Slave Station S

C1—Slave Overview (FIGS. 8-8D)

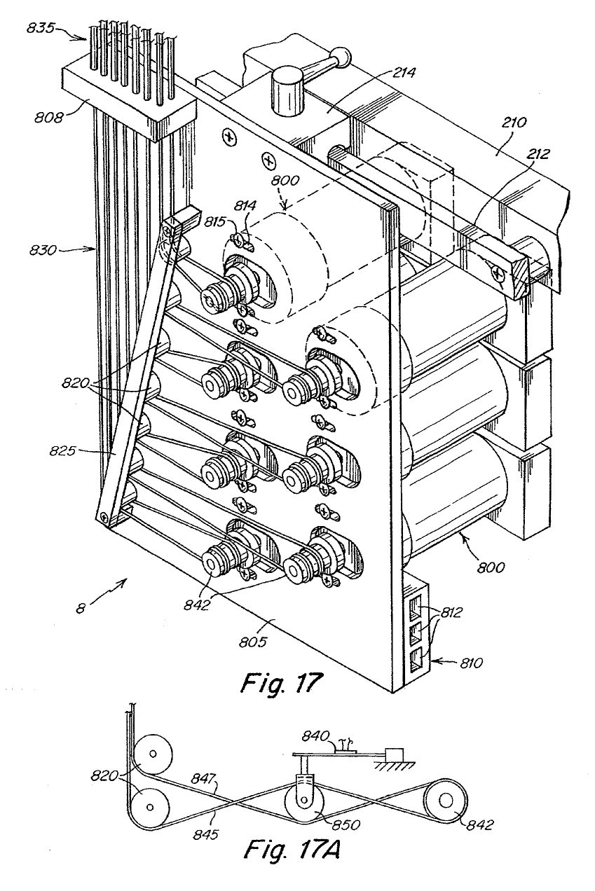

Reference is now made to FIG. 8 which is a perspective view illustrating the present embodiment of the slave station S. A section of the surgical tabletop T is shown, from which extends the rigid angled post 19 that supports the surgical instrument 14 at mounting bracket 25. The drive unit 8 is also supported from the side of the tabletop by an L-shaped brace 210 that carries an attaching member 212. The brace is suitably secured to the table T and the drive unit 8 is secured to the attaching member 212 by means of a clamp 214. A lower vertical arm 19A of the rigid support rod 19 is secured to the attaching member 212 by another clamping mechanism 216, which mechanism 216 permits vertical adjustment of the rigid support 19 and attached instrument 14. Horizontal adjustment of the surgical instrument is possible by sliding the mounting bracket 25 along an upper horizontal arm 19B of the support rod 19. One embodiment of the drive unit 8 is described in further detail in FIG. 17. A preferred embodiment is illustrated in FIGS. 8F-8L.

The clamping bracket 216 has a knob 213 that can be loosened to reposition the support rod 19 and tightened to hold the support rod 19 in the desired position. The support rod 19, at its vertical arm 19A, essentially moves up and down through the clamp 216. Similarly, the mounting bracket 25 can move along the horizontal arm 19B of the support rod 19, and be secured at different positions therealong. The clamp 214, which supports the drive unit 8 on the operating table, also has a knob 215 which can be loosened to enable the drive unit to be moved to different positions along the attaching member 212.

FIG. 8 also shows the cable-in-conduit bundles 21 and 22. The cables in the bundle 21 primarily control the action of the adapter or guide member 15. The cables in bundle 22 primarily control the tool 18, all described in further detail below.

FIG. 8 also illustrates a support yoke 220 to which is secured the mounting bracket 25, a pivot piece 222, and support rails 224 for a carriage 226. Piece 222 pivots relative to the support yoke 220 about pivot pin 225.

FIG. 2B is a schematic representation of the joint movements associated with the slave station S. The first joint movement J1 represents a pivoting of the instrument 14 about pivot pin 225 at axis 225A. The second joint movement J2 is a transition of the carriage 226 on the rails 224, which essentially moves the carriage and instrument 14 supported therefrom, in the direction indicated by the arrow 227. This is a movement toward and away from the operative site OS. Both of these movements J1 and J2 are controlled by cabling in bundle 21 in order to place the distal end of the guide tube 17 at the operative site. The operative site is defined as the general area in proximity to where movement of the tool 18 occurs, usually in the viewing area of the endoscope and away from the incision.

FIG. 8 also shows a coupler 230 pivotally coupled from a base piece 234 by means of a pivot pin 232. The coupler 230 is for engaging with and supporting the proximal end of the instrument insert 16.

Reference is now made to FIGS. 8A, 8B and 8C which are perspective views of a preferred clamping arrangement which allows a limited amount of pivoting of the mounting bracket 25 (which supports instrument 14). The mounting bracket 25 includes a securing knob 450 that clamps the mounting bracket 25 to a base 452. The mounting bracket is basically two pieces 455 and 457. A bottom piece 457 is adapted to receive the upper arm of rigid supporting rod 19 (see FIG. 8B) and is secured thereto by a bolt 458. A top piece 455 is pivotably adjustable relative to the bottom piece 457 by means of slots 460 that engage with bolts 462. When bolts 462 are loosened, the top piece 455 may be rotated relative to the bottom piece 457 so that the instrument 14 may be held in different positions. The bolts 462 may then be tightened when the instrument 14 is in a desired angular position.

An adjustable bracket 25 and support post 19 may be provided at each side of the table for mounting a surgical instrument 14 on both the left and the right sides of the table. Depending upon the particular surgical procedure, it may be desirable to orient a pair of guide tubes on the left and right sides in different arrangements. In the arrangement of FIG. 1, the guide tubes 17, 17 are arranged so that the respective tools 18, 18 face each other. However, for other procedures it may be desirable to dispose the guides in different positions, allowed by the adjustability of brackets 25, 25 on their respective support posts 19, 19.

FIG. 8D shows a template 470 useful in a preferred procedure for positioning the guide tube. In this procedure, when the support post 19 is initially positioned, the mounting bracket 25 holds the template 470 (rather than the instrument 14). The template 470 has a right angle arm 472 with a locating ball 474 at the end thereof. The arm 472 extends a distance that is substantially the same as the lateral displacement of the guide tube 200 from pivot point 205 above the incision point 207 in FIG. 2C (see also the trocar 487 at the incision point 485 in FIG. 2B). The mounting bracket 25 is adjusted on the support post 19 so that the ball 474 coincides with the intended incision point of the patient. Thereafter, the template is removed and when the instrument 14 is then clamped to the mounting bracket, the guide tube 17 will be in the proper position vis-a-vis the patient's incision. Thus, the template 470 is used to essentially position the bracket 25 where it is desired to be located with the ball 474 coinciding with the incision point. Once the template is removed and the instrument is secured, the guide tube 17 will be in the proper position relative to the incision.

In connection with the operation of the present system, once the patient is on the table, the drive unit 8 is clamped to the table. It's position can be adjusted along the table by means of the attaching member 212. The lower arm 19A of the rigid support rod 19 is secured to the table by the bracket 216. The surgeon determines where the incision is to be made. The mounting bracket on the rigid rod 19 is adjusted and the template 470 is secured to the clamp 25. The ball 474 on the template is lined up with the incision so as to position the securing rod 19 and clamp 25 in the proper position. At that time the rigid rod 19 and the securing clamp 25 are fixed in position. Then the template is removed and the instrument 14 is positioned on the clamp 25. The incision has been made and the guide tube 17 is inserted through the incision into the patient and the instrument 14 is secured at the fixed position of mounting bracket 25.

With regard to the incision point, reference is made to FIG. 2B which shows the incision point along the dashed line 485. Also shown at that point is the cannula 487. In some surgical procedures it is common to use a cannula in combination with a trocar that may be used to pierce the skin at the incision. The guide tube 17 may then be inserted through the flexible cannula so that the tool is at the operative site. The cannula typically has a port at which a gas such as carbon dioxide enters for insufflating the patient, and a switch that can be actuated to desufflate. The cannula may typically include a valve mechanism for preventing the escape of the gas.

C2—Slave Cabling and Decoupling (FIGS. 8E-8L)

FIG. 8E illustrates a mechanical cabling sequence at the slave station from the drive unit 8, through adaptor 15 and insert 16, to the tool 18. Reference will again be made to FIG. 8E after a description of further details of the slave station.

In the present embodiment the cable conduits 21 and 22 are detachable from the drive unit 8. This is illustrated in FIG. 8F wherein the drive unit includes separable housing sections 855 and 856. The instrument 14 along with the attached cable conduits 21 and 22 and housing section 856 are, as a unit, of relatively light weight and easily maneuverable (portable) to enable insertion of the instrument 14 into the patient prior to attachment to the bracket 25 on support post 19.

FIG. 8F is an exploded perspective view of the cable drive mechanism and instrument illustrating the de-coupling concepts of the present embodiment at the slave station S. A section of the surgical tabletop T which supports the rigid post 19 is shown. The drive unit 8 is supported from the side of the tabletop by an L-shaped brace 210 that carries an attaching member 212. The brace 210 is suitably secured to the table T. The drive unit 8 is secured to the attaching member 212 by means of a clamp 214. Similarly, the rigid support rod 19 is secured to the attaching member 212 by means of another clamping mechanism 216.

Also in FIG. 8F the instrument 14 is shown detached from (or not yet attached to) support post 19 at bracket 25. The instrument 14 along with cables 21 and 22 and lightweight housing section 856 provide a relatively small and lightweight decoupleable slave unit that is readily manually engageable (insertable) into the patient at the guide tube 17.

After insertion, the instrument assembly, with attached cables 21, 22 and housing 856, is attached to the support post 19 by means of the knob 26 engaging a threaded hole in base 452 of adapter 15. At the other end of the support post 19, bracket 216 has a knob 213 that is tightened when the support rod 19 is in the desired position. The support rod 19, at its vertical arm 19A, essentially moves up and down through the clamp 216. Similarly, the mounting bracket 25 can move along the horizontal arm 19B of the support rod to be secured at different positions therealong. A further clamp 214 enables the drive unit 8 to be moved to different positions along the attaching member 212.

FIG. 8F also shows the coupler 230 which is pivotally coupled from base piece 234 by means of the pivot pin 232. The coupler 230 is for engaging with and supporting the proximal end of the instrument insert 16.

Reference is now made to FIG. 8G which illustrates the mechanical cabling sequence at the slave station. The cabling extends from a motor 800 (of the drive unit 8), via adaptor 15, and via the instrument insert 16 to the tool 18. The adapter 15 and insert 16 are intercoupled by their associated interlocking wheels 324 and 334. Cables 606 and 607, which in reality, are a single-looped cable, extend between the interlocking wheel 334 and the tool 18. These cables 606, 607 are used for pivoting the wrist-joint mechanism (at the tool 18), in the direction of arrow J5 illustrated in FIG. 8G.

FIG. 8G also illustrates an idler pulley 344 on the insert 16, as well as a pair of pulleys 317 associated with the wheel 324 on the adapter 15. Cabling 315 extends from interlocking wheel 324 about the pulleys 317, about an idler pulley 318 and through sheathing 319 to conduit turn buckles 892. The cables 323 extending from the turn buckles 892 are wrapped about a coupler spindle 860. Associated with the coupler spindle 860 is a coupler disk 862 secured to output shaft of one of the motors 800 of drive unit 8.

Reference is now made to further cross-sectional views illustrated in FIGS. 8H-8L. FIG. 8H is a partially broken away front-elevational view as taken along line 8H-8H of FIG. 8F. FIGS. 8I and 8J are cross-sectional views taken respectively along lines 8I-8I and 8J-8J of FIG. 8H. FIG. 8K is a cross-sectional side view taken along line 8K-8K of FIG. 8H. Lastly, FIG. 8L is a cross-sectional view as taken along line 8L-8L of FIG. 8K.

These cross-sectional views illustrate a series of seven motors 800, one for each of an associated mechanical cabling assembly. In, FIG. 8K, there is illustrated one of the motors 800 with its output shaft 865 extending therefrom. The motor 800 is secured to a housing wall 866 (also shown in FIG. 8F). FIG. 8K also shows the angle iron 868 that is used to support the housing section 855 from the bracket 214 (see FIG. 8F).

A coupler disk 862 is illustrated in FIGS. 8J and 8K, secured to the shaft 865 by a set screw 869. The coupler disk 862 also supports a registration pin 871 that is adapted to be received in slots 873 of the coupler spindle 860. FIGS. 8K and 8L illustrate the pin 871 in one of the slots 873. The registration pin 871 is biased outwardly from the coupler disk by means of a coil spring 874.

The first housing section 855 also carries oppositely disposed thumb screws 875 (see FIG. 8H). These may be threaded through flanges 876 as illustrated in FIG. 8J. When loosened, these set screws enable the second housing section 856 to engage with the first housing section 855. For this purpose, there is provided a slot 878 illustrated in FIG. 8F. Once the second housing section 856 is engaged with the first housing section 855, then the thumb screws 875 may be tightened to hold the two housing sections together, at the same time facilitating engagement between the coupler disks 862 and the coupler spindles 860.

The cross-sectional view of FIG. 8K shows that at the end of coupler disk 862 where it is adapted to engage with the coupler spindle 860, the coupler disk is tapered as illustrated at 879. This facilitates engagement between the coupler disk and the coupler spindle.

As illustrated in FIG. 8F, the two housing sections 855 and 856 are separable from each other so that the relatively compact slave unit can be engaged and disengaged from the motor array, particularly from the first housing section 855 that contains the motors 800. The first housing section 855, as described previously, contains the motors 800 and their corresponding coupler disks 862. In FIG. 8F, the second housing section 856 primarily accommodates and supports the coupler spindles 860 and the cabling extending from each of the spindles to the cable bundles 21 and 22 depicted in FIG. 8F.

FIGS. 8J and 8K illustrate one of the coupler spindles 860 supported within a pair of bearings 881. The cable associated with the coupler spindle is secured to the coupler spindle by means of a cable clamp screw 883. FIGS. 8J and 8K illustrate the cable extending about the coupler spindle, and secured by the cable clamp screw 883. The particular cable illustrated in FIG. 8J about spindle 860 is identified as cable D.

In FIGS. 8H-8K, the cabling is identified by cables A-G. This represents seven separate cables that are illustrated, for example, in FIG. 8H as extending into the second housing section 856 with a flexible boot 885 (see the top of FIGS. 8H and 8K) extending thereabout.

At the top of the second housing section 856 there is provided a conduit stop or retainer 888 that is secured in place at the top of the housing section in an appropriate manner. The conduit retainer 888 has through slots 890, one for accommodating each of the cables A-G (see FIG. 8I). Refer in particular to FIGS. 8H and 8K illustrating the cables A-G extending through the retainer 888 in the slots thereof. Each of the cables may also be provided with a turnbuckle 892 that is useful in tensioning the cables. Each turnbuckle 892 screws into an accommodating threaded passage in the retainer 888, as illustrated in FIG. 8K.

In FIG. 8H the coupler spindles are all disposed in a linear array. To properly accommodate the cabling, the spindles are of varying diameter, commencing at the top of the second housing section 856 with the smallest diameter spindle and progressing in slightly larger diameter spindles down to the bottom of the second housing section 856 where there is disposed the largest diameter coupler spindle.

The detachability of the two housing sections 855 and 856 enables the cleaning of certain components which are disposed above the plane of the operating table, here referred to as the sterile field. More specifically, the detachable housing 856 with attached cables 21 and 22 and instrument 14, needs to be sterilized after use, except for the instrument insert 16 which is an integral disposal unit. The sterilization of the designated components may include a mechanical cleaning with brushes or the like in a sink, followed by placement in a tray and autoclave in which the components are subjected to superheated steam to sterilize the same. In this manner, the adapter 15 is reusable. Also, the engagement between the adapter 15 and insert 16 is such that the disposable insert element may have holes, which are relatively hard to clean, whereas the recleanable adapter element has a minimum number of corresponding projections, which are relatively easier to clean than the holes. By disposable, it is meant that the unit, here the insert 16, is intended for a single use as sold in the marketplace. The disposable insert interfaces with an adapter 15 which is intended to be recleaned (sterilized) between repeated uses. Preferably, the disposable unit, here the insert 16, can be made of relatively lower cost polymers and materials which, for example, can be molded by low-cost injection molding. In addition, the disposable instrument insert 16 is designed to require a relatively minimal effort by the operator or other assistant who is required to attach the insert to the adapter 15. More specifically, the operator is not required to rethread any of the multiple mechanical cabling assemblies.

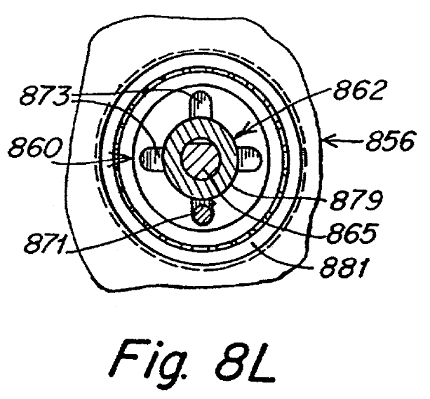

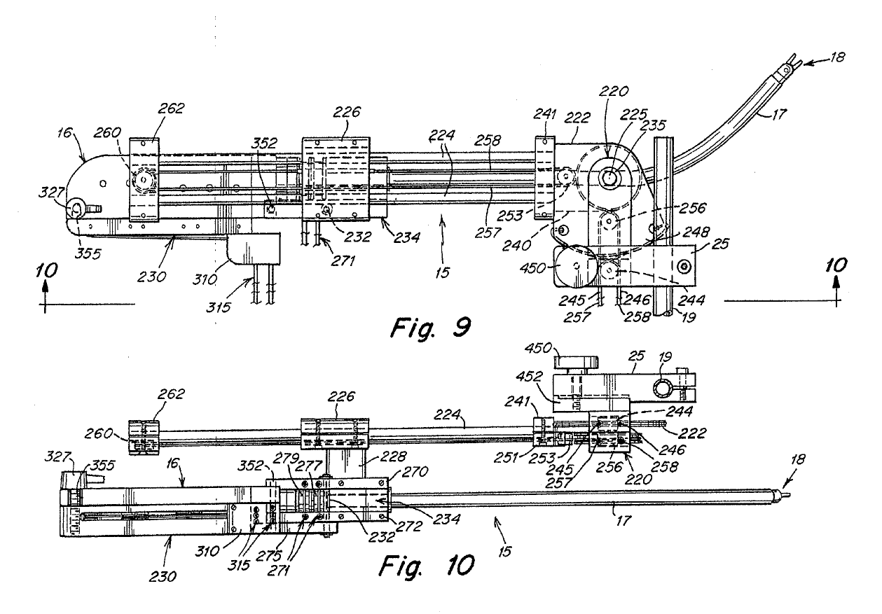

C3—Slave Instrument Assembly (FIGS. 9-16)

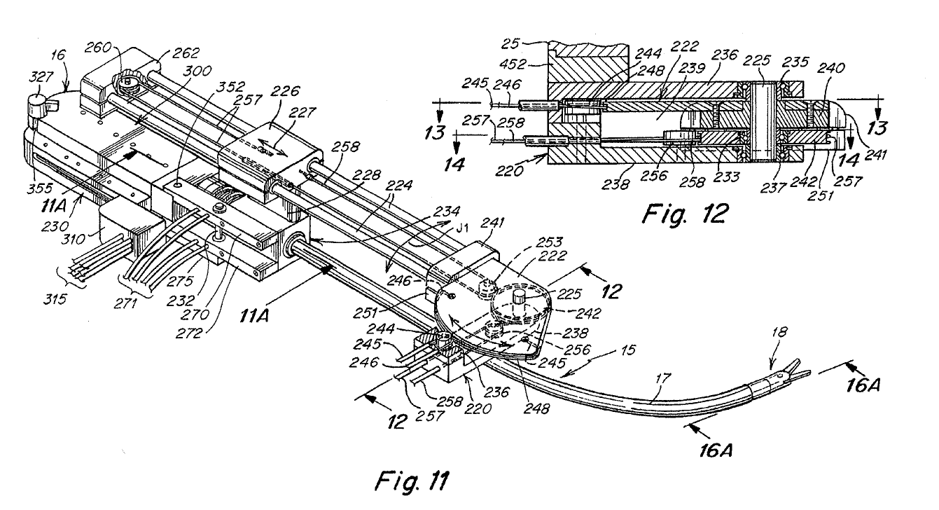

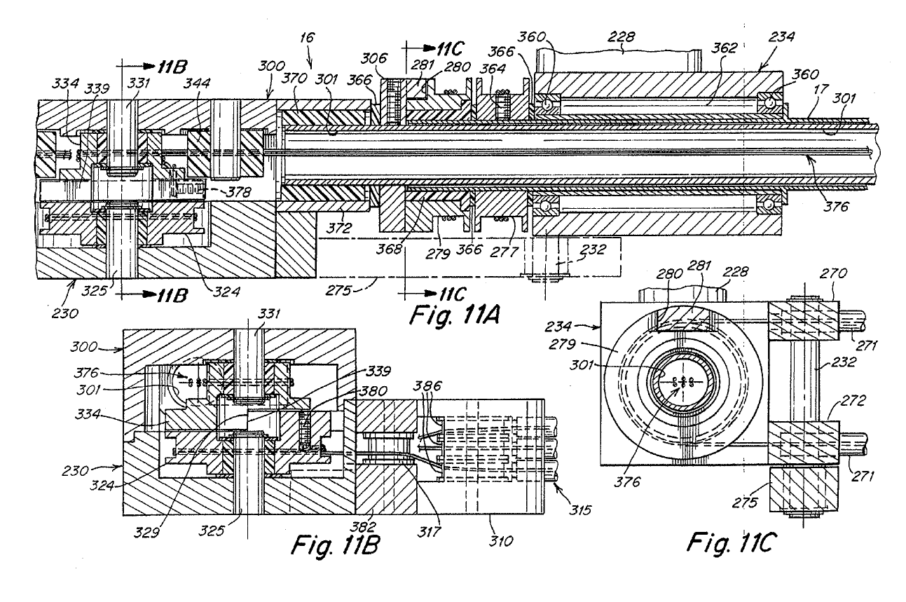

Further details of the detachable and portable slave unit are shown in FIGS. 9-16. For example, FIG. 11 shows the carriage 226 which extends from the mounting bracket 25 on support post 19. Below carriage 226, a base piece 234 is supported from the carriage 226 by a rectangular post 228. The post 228 supports the entire instrument assembly, including the adaptor 15 and the instrument insert 16 once engaged.

As indicated previously, a support yoke 220 is supported in a fixed position from the mounting bracket 25 via base 452. Cabling 21 extends into the support yoke 220. The support yoke 220 may be considered as having an upper leg 236 and a lower leg 238 (see FIG. 12). In the opening 239 between these legs 236, 238 there is arranged the pivot piece 222 with its attached base 240. Below the base 240 and supported by the pivot pin 225 is a circular disc 242 that is stationary relative to the yoke legs 236, 238. A bearing 235 in leg 236, a bearing 237 in leg 238, and a bearing 233 in disc 242, allow rotation of these members relative to the pivot pin 225.

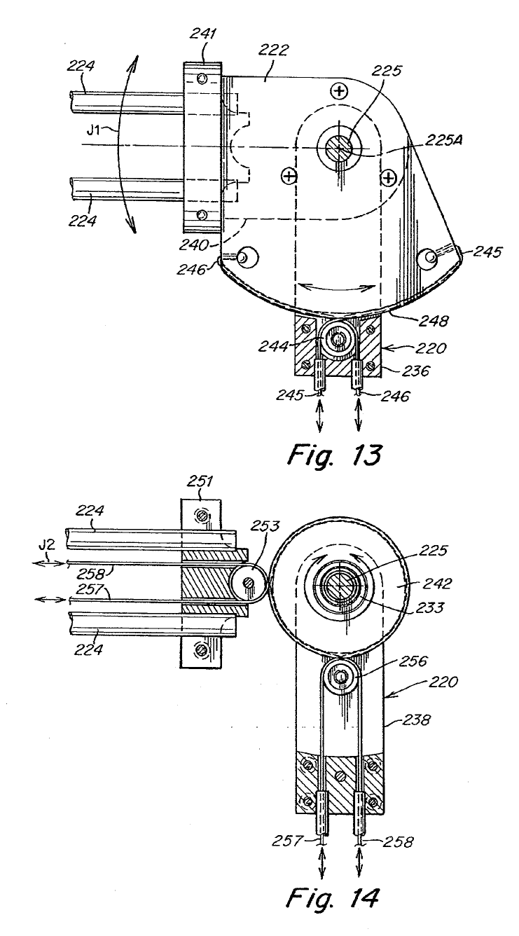

Disposed within a recess in the support yoke 220, as illustrated in FIG. 13, is a capstan 244 about which cables 245 and 246 extend and are coupled to opposite sides of the arcuate segment 248 of pivot piece 222. The ends of cables 245 and 246 are secured in holes at opposite sides of arcuate segment 248. The cables 245 and 246 operate in conjunction with each other. At their other ends, these cables connect to a motor. Depending upon the direction of rotation of the motor, either cable 245 or cable 246 will be pulled, causing the pivot price 232 to rotate in a direction indicated by J1.

The base 240 of pivot piece 222 also has at one end thereof an end piece 241 into which are partially supported the ends of rails 224 (see FIG. 13). The other ends of the rails are supported by an end piece 251, which also has cabling 257, 258 for the carriage 226 extending therethrough, such as illustrated in FIG. 14. A capstan 253 is supported from a lower surface of the base 240. Another capstan 256 is supported within the support yoke 220. The cables 257 and 258 extend about the capstan 256, about disc 242 (which may be grooved to receive the cables), to the carriage 226, and from there about another capstan 260 disposed within end member 262 (see FIG. 11). End member 262 supports the other ends of the rails 224, upon which the carriage 226 transitions. The ends of the cables 257 and 258 are secured appropriately within the carriage. FIG. 11 illustrates by the arrow 227 the forward and backward motion of the carriage 226, and thus of the attached actuator 15 toward and away from the operative site.

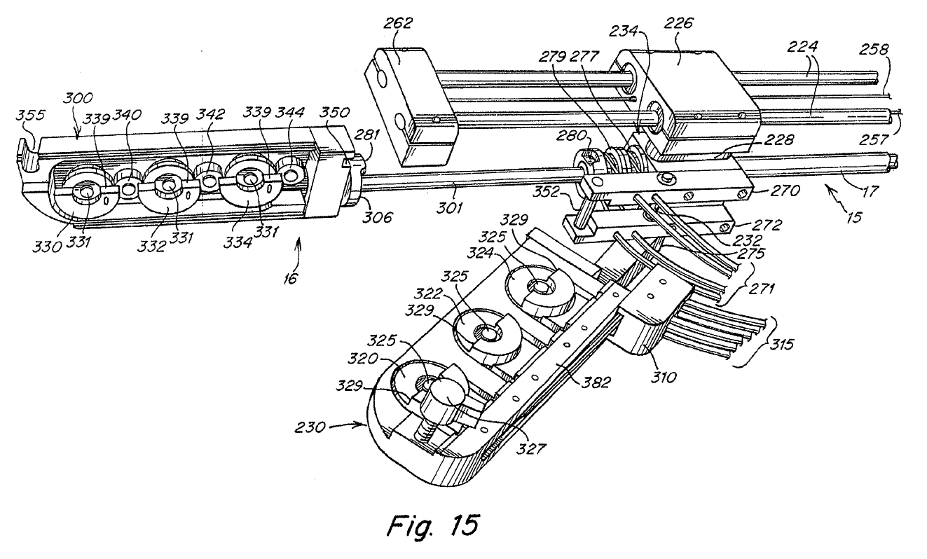

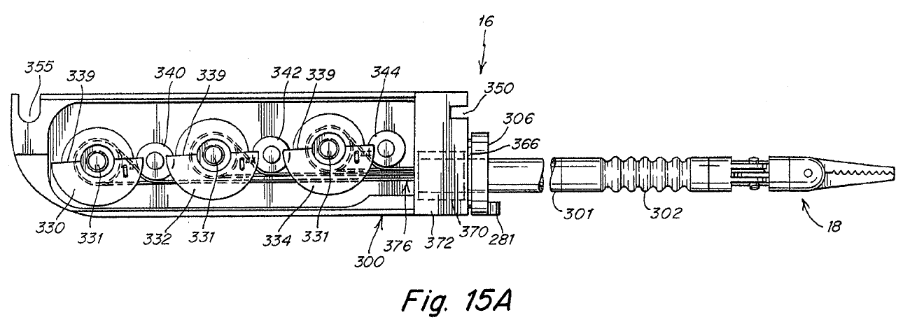

Now, reference is made to FIG. 15 illustrating a portion of the slave unit with the instrument insert 16 partially removed and rotated from the base piece 234. FIG. 15 shows a portion of the carriage mechanism, including the carriage 226 supported on rails 224. As indicated previously, below the carriage 226 there is a support post 228 that supports the base piece 234. It is at the base piece 234, that cabling 22 from the drive unit 8 is received.

Also extending from the base piece 234 is the guide tube 17 of adapter 15. The guide tube 17 accommodates, through its center axial passage, the instrument insert 16. Also, supported from the base piece 234, at pivot pin 232, is the adaptor coupler 230. The adaptor coupler 230 pivots out of the way so that the instrument insert 16 can be inserted into the adaptor 15. FIG. 15 shows the instrument insert 16 partially withdrawn from the adaptor 15. The pivot pin 232 may be longer than the distance between the two parallel bars 270 and 272 carried by base piece 234, so that the pin not only allows rotation, but can also slide relative to bars 270 and 272. This permits the coupler 230 to not only pivot, but also to move laterally to enable better access of the instrument insert 16 into the base piece 234. The instrument insert 16 has a base (coupler) 300 that in essence is a companion coupler to the adapter coupler 230.

With further reference to FIG. 15, the instrument insert 16 is comprised of a coupler 300 at the proximal end, and at the distal end an elongated shaft or stem, which in this embodiment has a more rigid proximal stem section 301 and a flexible distal stem section 302 (see FIG. 15A). The distal stem section 302 carries the tool 18 at its distal end. The instrument coupler 300 includes one or more wheels 339 which laterally engage complimentary wheels 329 of the coupler 230 on adaptor 15. The instrument coupler 300 also includes an axial wheel 306 at its distal end through which the stem 301 extends, and which also engages a wheel on the adaptor, as to be described below in further detail. The axial engagement wheel 306 is fixed to the more rigid stem section 301, and is used to rotate the tool 18 axially at the distal end of the flexible stem section 302 (as shown by arrow J4 in FIG. 2B).

The base piece 234 has secured thereto two parallel spaced-apart bars 270 and 272. It is between these bars 270 and 272 that is disposed the pivot pin 232. The pivot pin 232 may be supported at either end in bearings in the bars 270 and 272, and as previously mentioned, has limited sliding capability so as to move the adapter coupler 230 away from base piece 234 to enable insertion of the instrument insert 16. A leg 275 is secured to the pivot pin 232. The leg 275 extends from the coupler 230 and provides for pivoting of coupler 230 with respect to base piece 234. Thus, the combination of pivot pin 232 and the leg 275 permits a free rotation of the coupler 230 from a position where it is clear to insert the instrument insert 16 to a position where the coupler 230 intercouples with the base 300 of the instrument insert 16. As depicted in FIG. 15, the bars 270 and 272 also accommodate therethrough cabling from cable bundles 271.

The base piece 234 also rotatably supports the rigid tube 17 (illustrated by arrow J3 in FIG. 2B). As indicated previously, it is the connection to the carriage 226 via post 228 that enables the actuator 15 to move toward and away from the operative site. The rotation of the tube 17 is carried out by rotation of pulley 277 (see FIG. 15). A pair of cables from the bundle 271 extend about the pulley 277 and can rotate the pulley in either direction depending upon which cable is activated. To carry out this action, the tube 17 is actually supported on bearings within the base piece 234. Also, the proximal end of the tube 17 is fixed to the pulley 277 so that the guide tube 17 rotates with the pulley 277.

Also supported from the very proximal end of the tube 17, is a second pulley 279 that is supported for rotation about the actuator tube 17. For this purpose a bearing is disposed between the pulley 279 and the actuator tube. The pulley 279 is operated from another pair of cables in the bundle 271 that operate in the same manner. The cabling is such that two cables couple to the pulley 279 for operation of the pulley in opposite directions. Also, as depicted in FIG. 15, the pulley 279 has a detent at 280 that is adapted to mate with a tab 281 on the axial wheel 306 of instrument coupler 300. Thus, as the pulley 279 is rotated, this causes a rotation of the axial wheel 306 and a corresponding rotation of flexible and rigid sections 301, 302 of the instrument insert 16, including the tool 18.

Again referring to FIG. 15, a block 310 is secured to one side of the coupler 230. The block 310 is next to the leg 275 and contains a series of small, preferably plastic, pulleys that accommodate cabling 315. These cables extend to other pulleys 317 disposed along the length of the coupler 230. Refer also to the cabling diagram of FIG. 8E.

In this embodiment, the coupler 230 includes wheels 320, 322 and 324. Each of these wheels is provided with a center pivot 325 to enable rotation of the wheels in the coupler 230. The knob 327 is used to secure together the adapter coupler 230 and the base coupler 300 of the instrument insert 16.

For the three wheels, 320, 322 and 324, there are six corresponding pulleys 317, two pulleys being associated with each wheel (see FIGS. 8E and 11B). Similarly, there are six pulleys in the block 310. Thus, for cabling bundle 315 there are six separate cable conduits for the six separate cables that couple to the wheels 320, 322 and 324. Two cables connect to each wheel for controlling respective opposite directions of rotation thereof.

Each of the wheels 320, 322 and 324 have a half-moon portion with a flat side 329. Similarly, the instrument base 300 has companion wheels 330, 332 and 334 with complimentary half-moon construction for engagement with the wheels 320, 322 and 324. The wheel 320 controls one of the jaws of the tool 18 (motion J6 in FIG. 2B). The wheel 324 controls the other jaw of the tool 18 (motion J7 in FIG. 2B). The middle wheel 322 controls the wrist pivoting of the tool 18 (motion J5 in FIG. 2B). Also refer to FIG. 8E showing cabling for controlling tool movement.

The coupler 300 of insert 16 has three wheels 330, 332 and 334, each with a pivot pin 331, and which mate with the corresponding wheels 320, 322 and 324, respectively of the adaptor coupler. In FIG. 15 the instrument base piece 300 is shown rotated from its normal position for proper viewing of the wheels. Normally, it is rotated through 180.degree. so that the half-moon wheels 330, 332 and 334 engage with the corresponding coupler wheels 320, 322 and 324. Also illustrated in FIG. 15 are capstans or idler pulleys 340, 342 and 344 associated, respectively, with wheels 330, 332 and 334.

As shown in FIG. 15A, each wheel of the instrument coupler 300 has two cables 376 that are affixed to the wheel (e.g., wheel 334 in FIG. 8E) and wrapped about opposite sides at its base. The lower cable rides over one of the idler pulleys or capstans (e.g., capstan 34 in FIG. 8E), which routes the cables toward the center of the instrument stem 301. It is desirable to maintain the cables near the center of the instrument stem. The closer the cables are to the central axis of the stem, the less disturbance motion on the cables when the insert stem is rotated. The cables may then be routed through fixed-length plastic tubes that are affixed to the proximal end of the stem section 301 and the distal end of the stem section 302. The tubes maintain constant length pathways for the cables as they move within the instrument stem.

The instrument coupler 300 is also provided with a registration slot 350 at its distal end. The slot 350 engages with a registration pin 352 supported between the bars 270 and 272 of base piece 234. The coupler 300 is also provided with a clamping slot 355 on its proximal end for accommodating the threaded portion of the clamping knob 327 (on adapter coupler 230). The knob 327 affirmatively engages and interconnects the couplers 230 and 300.

In operation, once the surgeon has selected a particular instrument insert 16, it is inserted into the adapter 15. The proximal stem 301, having the distal stem 302 and the tool 18 at the distal end, extend through the adapter guide tube 17. FIG. 8 shows the tool 18 extending out of the guide tube 17 when the surgical instrument 16 is fully inserted into the adaptor 15. When it is fully inserted, the tab 281 on the axial wheel 306 engages with the mating detent 280 in pulley 279. Also, the registration slot 350 engages with the registration pin 352. Then the coupler 230 is pivoted over the base 300 of the instrument insert 16. As this pivoting occurs, the respective wheels of the coupler 230 and the coupler 300 interengage so that drive can occur from the coupler 230 to the insert 16. The knob 327 is secured down so that the two couplers 230 and 300 remain in fixed relative positions.

Reference is also now made to detailed cross-sectional views of FIGS. 11A, 11B and 11C. FIG. 11A is a cross sectional view taken along line 11A-11A of FIG. 11. FIG. 11B is a cross-sectional view taken along line 11B-11B of FIG. 11A. FIG. 11C is a further cross-sectional view taken through FIG. 11A along line 11C-11C.

The base piece 234 of adapter 15 rotatably supports the guide tube 17, allowing rotation J3 shown in FIG. 2B. As noted in FIG. 11A, there are a pair of bearings 360 disposed at each end within the axial passage 362 in the base piece 234. The rotation of the guide tube 17 is carried out by rotation of the first pulley 277. In FIG. 11A there is a set screw 364 that secures the pulley 277 to the guide tube 17. Nylon spacers 366 separate various components, such as the base piece 234 and the pulley 277, the two pulleys 277 and 279, and base 300 and wheel 306.

A nylon bearing 368 is also provided between the second pulley 279 and the guide tube 17. FIG. 11A also shows the proximal stem section 301 of the insert 16 inside of the guide tube 17. A nylon bearing 370 is supported within the front block 372 of the insert 16.

In FIG. 11A, the second pulley 279 is supported from the proximal end of the tube 17. The bearing 368 is disposed between the pulley 279 and the tube 17. The pulley 279 has a detent 280 that is adapted to mate with a tab 281 on the axial wheel 306. Thus, when the pulley 279 is rotated by cabling 271 (see FIG. 11C), this causes a rotation of the axial wheel 306, and a corresponding rotation (motion J4 in FIG. 2B) of the sections 301, 302 of the instrument insert 16, including the tool 18. The very proximal end of the section 301 is illustrated in FIG. 11A as being rotatable relative to the bearing 370.

FIG. 11A also shows the intercoupling of the instrument and adapter couplers 230 and 300. Here wheel 324 is shown interlocked with wheel 334. FIGS. 11A and 11C also show cabling at 376. This cabling includes six separate cables that extend through the length of the stem 301, 302 of the instrument. The cabling is illustrated connecting about an idler pulley 344. The cabling associated with wheel 334 is secured by the cable clamping screw 378. For further details of the cabling, refer to FIG. 8E.

FIG. 11B is a cross-sectional view taken along 11B-11B of FIG. 11A which again shows the cooperating wheels 324 and 334. Also illustrated is a cable clamping set screw 380 that is used to secure the cabling 376 to wheel 324. A cable guide rail 382 is attached and forms part of the base of the adapter coupler 230. The cable guide rail 382 contains six idler pulleys 317, one of which is illustrated in FIG. 11B. It is noted that cabling 376 extends about this pulley to the cable idler block 310 where conduits 315 are coupled. The cable guide idler block 310 includes a series of six idler pulleys shown in dotted outline in FIG. 11B at 386.

FIG. 11C is a cross-sectional view taken along line 11C-11C of FIG. 11A, which shows further details at the pulley 279. Also illustrated is post 228 supporting the base piece 234 of the instrument insert, and cabling 376 extending through the instrument.

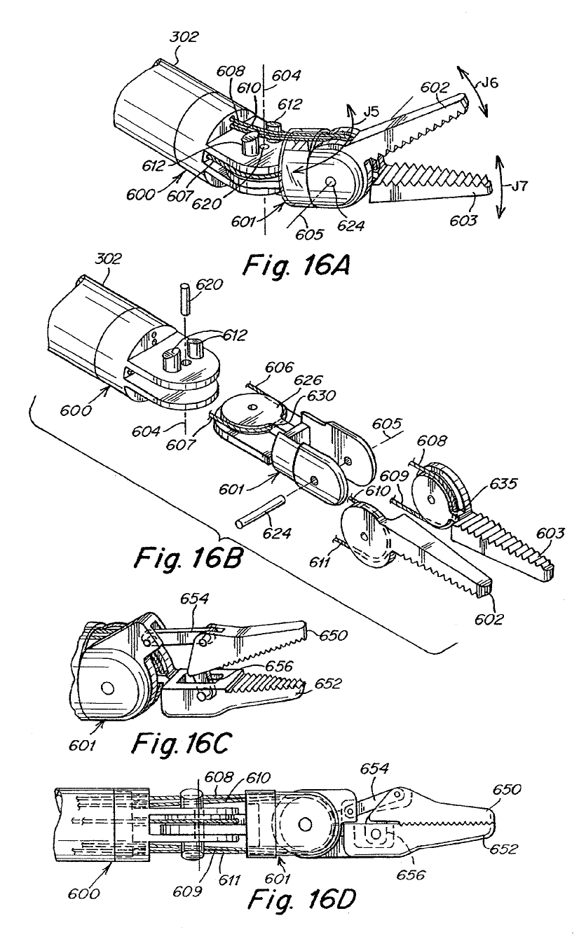

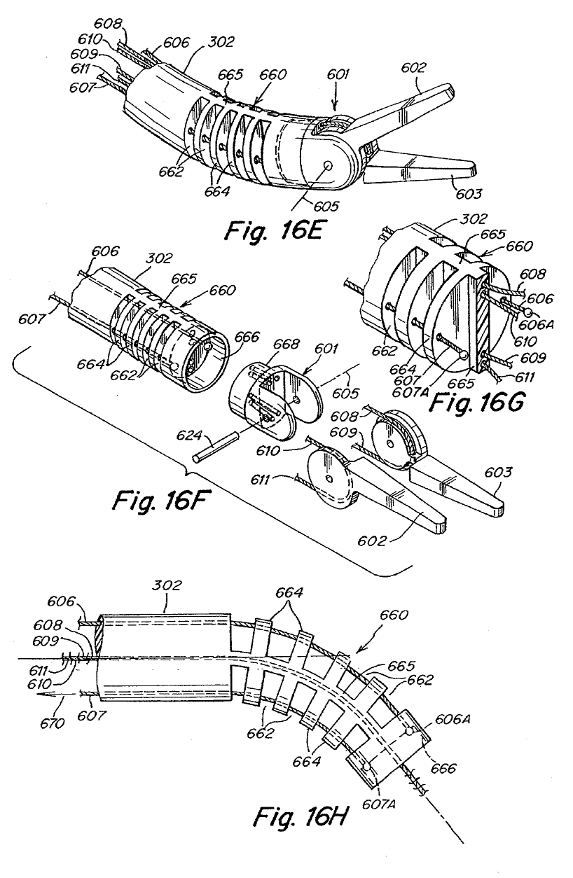

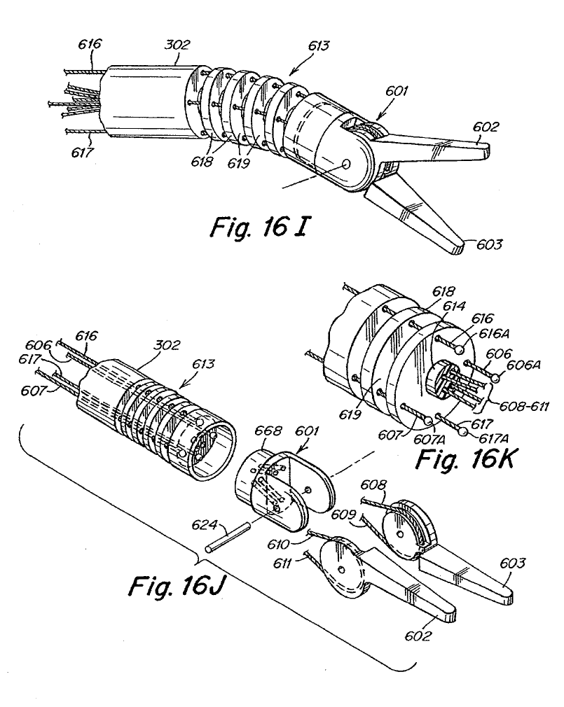

FIGS. 16A and 16B illustrate the construction of one form of a tool. FIG. 16A is a perspective view and FIG. 16B is an exploded view. The tool 18 is comprised of four members including a base 600, link 601, upper grip or jaw 602 and lower grip or jaw 603. The base 600 is affixed to the flexible stem section 302 (see FIG. 15A). The flexible stem may be constructed of a ribbed plastic. This flexible section is used so that the instrument will readily bend through the curved part of the guide tube 17.

The link 601 is rotatably connected to the base 600 about axis 604. FIG. 16B illustrates a pivot pin 620 at axis 604. The upper and lower jaws 602 and 603 are rotatably connected by pivot pin 624 to the link 601 about axis 605, where axis 605 is essentially perpendicular to axis 604.

Six cables 606-611 actuate the four members 600-603 of the tool. Cable 606 travels through the insert stem (section 302) and through a hole in the base 600, wraps around curved surface 626 on link 601, and then attaches on link 601 at 630. Tension on cable 606 rotates the link 601, and attached upper and lower grips 602 and 603, about axis 604 (motion J5 in FIG. 2B). Cable 607 provides the opposing action to cable 606, and goes through the same routing pathway, but on the opposite sides of the insert. Cable 607 may also attach to link 601 generally at 630.

Cables 608 and 610 also travel through the stem 301, 302 and though holes in the base 600. The cables 608 and 610 then pass between two fixed posts 612. These posts constrain the cables to pass substantially through the axis 604, which defines rotation of the link 601. This construction essentially allows free rotation of the link 601 with minimal length changes in cables 608-611. In other words, the cables 608-611, which actuate the jaws 602 and 603, are essentially decoupled from the motion of link 601. Cables 608 and 610 pass over rounded sections and terminate on jaws 602 and 603, respectively. Tension on cables 608 and 610 rotate jaws 602 and 603 counter-clockwise about axis 605. Finally, as shown in FIG. 16B, the cables 609 and 611 pass through the same routing pathway as cables 608 and 610, but on the opposite side of the instrument. These cables 609 and 611 provide the clockwise motion to jaws 602 and 603, respectively. At the jaws 602 and 603, as depicted in FIG. 16B, the ends of cables 608-611 may be secured at 635, for example by the use of an adhesive such as epoxy glue, or the cables could be crimped to the jaws.

To review the allowed movements of the various components of the slave unit, the instrument insert 16 slides through the guide tube 17 of adaptor 15, and laterally engages the adaptor coupler 230. The adaptor coupler 230 is pivotally mounted to the base piece 234. The base piece 234 rotationally mounts the guide tube 17 (motion J3). The base piece 234 is affixed to the linear slider or carriage assembly (motion J2). The carriage assembly in turn is pivotally mounted at the pivot 225 (motion J1).

Reference is now made to FIGS. 16C and 16D. FIG. 16C is a fragmentary perspective view of an alternate set of jaws, referred to as needle drivers. FIG. 16D is a side elevation view of the needle drivers. This embodiment employs an over-center camming arrangement so that the jaw is not only closed, but also at a forced closure.

In FIGS. 16C and 16D, similar reference characters are employed with respect to the embodiment of FIGS. 16A and 16B. Thus, there is provided a base 600, a link 601, an upper jaw 650 and a lower jaw 652. The base 600 is affixed to the flexible stem section 302. Cabling 608-611 operate the end jaws. Linkages 654 and 656 provide the over-center camming operation.

The two embodiments of FIGS. 16A-16D employ a fixed wrist pivot. An alternate construction is illustrated in FIGS. 16E-16H in which there is provided, in place of a wrist pivot, a flexible or bending section. In FIGS. 16E-16H, similar reference characters are used for many of the parts, as they correspond to elements found in FIGS. 16A-16D.

In the embodiment of FIGS. 16E-16H, the tool 18 is comprised of an upper grip or jaw 602 and a lower grip or jaw 603, supported from a link 601. Each of the jaws 602,603, as well as the link 601, may be constructed of metal, or alternatively, the link 601 may be constructed of a hard plastic. The link 601 is engaged with the distal end of the flexible stem section 302. In this regard reference may also be made to FIG. 15A that shows the ribbed, plastic construction of the flexible stem section 302. FIG. 16E shows only the very distal end of the stem section 302, terminating in a bending or flexing section 660. The flexible stem section 302 is constructed so as to be flexible and thus has a substantial length of a ribbed surface as illustrated in FIG. 15A. Also, at the flexible section 660, flexibility and bending is enhanced by means of diametrically-disposed slots 662 that define therebetween ribs 664. The flexible section 660 also has a longitudinally extending wall 665, through which cabling extends, particularly for operation of the tool jaws. The very distal end of the bending section 660 terminates with an opening 666 for receiving the end 668 of the link 601. The cabling 608-611 is preferably at the center of the flex section at wall 665 so as to effectively decouple flex or bending motions from tool motions.

Regarding the operation of the tool, reference is made to the cables 608, 609, 610, and 611. All of these extend through the flexible stem section and also through the wall 665 such as illustrated in FIG. 16G. The cables extend to the respective jaws 602,603 for controlling operation thereof in a manner similar to that described previously in connection with FIGS. 16A-16D. FIGS. 16E-16H also illustrate the cables 606 and 607 which couple through the bending section 660 and terminate at ball ends 606A and 607A, respectively. Again, refer to FIG. 16G that shows these cables. FIGS. 16F and 16H also show the cables 606, 607 with the ball ends 606A, 607A, respectively. These ball ends are adapted to urge against the very end of the bendable section in opening 666. When these cables are pulled individually, they can cause a bending of the wrist at the bending or flexing section 660. FIG. 16H illustrates the cable 607 having been pulled in the direction of arrow 670 so as to flex the section 660 in the manner illustrated in FIG. 16H. Pulling on the other cable 606 causes a bending in the opposite direction.

By virtue of the slots 662 forming the ribs 664, there is provided a structure that bends quite readily, essentially bending the wall 665 by compressing at the slots such as in the manner illustrated in FIG. 16H. This construction eliminates the need for a wrist pin or hinge.

The embodiment illustrated in FIG. 16F has a separate link 601. However, in an alternate embodiment, this link 601 may be fabricated integrally with, and as part of, the bending section 660. For this purpose the link 601 would then be constructed of a relatively hard plastic rather than the metal link as illustrated in FIG. 16F and would be integral with section 660.

In another embodiment, the bending or flexing section 660 can be constructed so as to have orthogonal bending by using four cables separated at 90.degree. intervals and by providing a center support with ribs and slots about the entire periphery. This embodiment is shown in FIGS. 16I-16K. The bending section 613 is at the end of flexible stem section 302. The cables 608,609,610 and 611 are for actuation of the jaws 602 and 603 in the same manner as for earlier embodiments. The link 601 couples the bending section 613 to the jaws 602 and 603.

The bending section has a center support wall 614 supporting ribs 618 separated by slots 619. This version enables bending in orthogonal directions by means of four cables 606,607,616 and 617, instead of the single degree-of-freedom of FIG. 16E. The operation of cables 606 and 607 provides flexing in one degree-of-freedom, while an added degree-of-freedom is provided by operation of cables 616 and 617.

Mention has also been made of various forms of tools that can be used. The tool may comprise a variety of articulated tools such as: jaws, scissors, graspers, needle holders, micro dissectors, staple appliers, tackers, suction irrigation tools and clip appliers. In addition, the tool may comprise a non-articulated instrument such as: a cutting blade, probe, irrigator, catheter or suction orifice.

C4—Slave Drive Unit (FIGS. 17-17A)

Reference is now made to the perspective view of the drive unit 8, previously illustrated in FIG. 8. FIG. 17 illustrates the drive unit 8 with the cover removed. The drive unit is adjustably positionable along rail 212 by an angle brace 210 that is attached to the operating table. Within the drive unit 8 are seven separate motors 800, corresponding to the seven separate controls at the slave station, and more particularly, to motions J1-J7 previously described in reference to FIG. 2B.

The drive unit includes a support plate 805 to which there is secured a holder 808 for receiving and clamping the cabling conduits 835. The motors 800 are each supported from the support plate 805. FIG. 17 also illustrates the electrical interface at 810, with one or more electrical connectors 812.

Regarding support for the motors 800 there is provided, associated with each motor, a pair of opposed adjusting slots 814 and adjusting screws 815. This permits a certain degree of positional adjustment of the motors, relative to their associated idler pulleys 820. The seven idler pulleys are supported for rotation by means of a support bar 825. FIG. 17 also shows the cabling coming 830 from each of the idler pulleys. With seven motors, and two cables coming off of each motor for opposite direction control, there are a total of fourteen separate cables conduits at the bundle 835. The cables move within the conduits in a known manner. The conduits themselves are fixedly supported and extend from the holder 808 to the adapter 15. Again, reference may be made to FIG. 8 showing the conduit bundles at 21 and 22.

The seven motors in this embodiment control (1) one jaw of the tool J6, (2) the pivoting of the wrist at the tool J5, (3) the other jaw of the tool J7, (4) rotation of the insert J4, (5) rotation of the adaptor J3, (6) linear carriage motion J2 and (7) pivoting of the adaptor J1. Of course, fewer or lesser numbers of motors may be provided in other embodiments and the sequence of the controls may be different.

FIG. 17A illustrates another aspect of the invention-a feedback system that feeds force information from the slave station back to the master station where the surgeon is manipulating the input device. For example, if the surgeon is moving his arm to the left and this causes some resistance at the slave station, the resistance is detected at the slave station and coupled back to one of the motors at the master station to drive the input device, such as the hand assembly illustrated herein, back in the opposite direction. This provides an increased resistance to the surgeon's movements which occurs substantially instantaneously.

FIG. 17A illustrates schematically a load cell 840 that is adapted to sense cable tension. FIG. 17A shows one of the pulleys 842 associated with one of the motors 800, and cables 845 and 847 disposed about a sensing pulley 850. The sensing pulley 850 is coupled to the piezoelectric load cell 840. The load cell 840 may be disposed in a Wheatstone bridge arrangement.

Thus, if one of the motors is operating under tension, this is sensed by the load cell 840 and an electrical signal is coupled from the slave station, by way of the controller 9, to the master station to control one of the master station motors. When tension is sensed, this drives the master station motor in the opposite direction (to the direction of movement of the surgeon) to indicate to the surgeon that a barrier or some other obstacle has been encountered by the element of the slave unit being driven by the surgeon's movements.

The cabling scheme is important as it permits the motors to be located in a position remote from the adaptor and insert. Furthermore, it does not require the motor to be supported on any moving arms or the like. Several prior systems employing motor control have motors supported on moveable arms. Here the motors are separated from the active instrument area (and sterile field) and furthermore are maintained fixed in position. This is illustrated in FIG. 8E by the motor 800. FIG. 8E also illustrates a typical cabling sequence from the motor 800 through to the tool 18. Both ends of cabling 315 are secured to the motor at 842 and the motor is adapted to rotate either clockwise or counterclockwise, in order to pull the cabling in either one direction or the other. The pair of cabling operates in unison so that as one cable is pulled inwardly toward the motor, the other cable pulls outwardly. As illustrated in FIG. 8E, the cables extend over pulley 820 to other pulleys, such as the pair of pulleys 317 and control wheel 324 associated with coupler 230. From there, the mechanical drive is transferred to the control wheel 334 of the instrument insert 300, which is coupled to wheel 334 and to the output cables 606 and 607 which drive wrist rotation of the tool 18, identified in FIG. 8E by the motion J5.

Another important aspect is the use of inter-mating wheels, such as the wheels 324 and 334 illustrated in FIG. 8E. This permits essentially a physical interruption of the mechanical cables, but at the same time a mechanical drive coupling between the cables. This permits the use of an instrument insert 16 that is readily engageable with the adaptor, as well as disengagable from the adaptor 15. This makes the instrument insert 16 easily replacable and also, due to the simplicity of the instrument insert 16, it can be made disposable. Refer again to FIG. 15A which shows the complete instrument insert and its relatively simple construction, but which still provides an effective coupling between the drive motor and the tool.

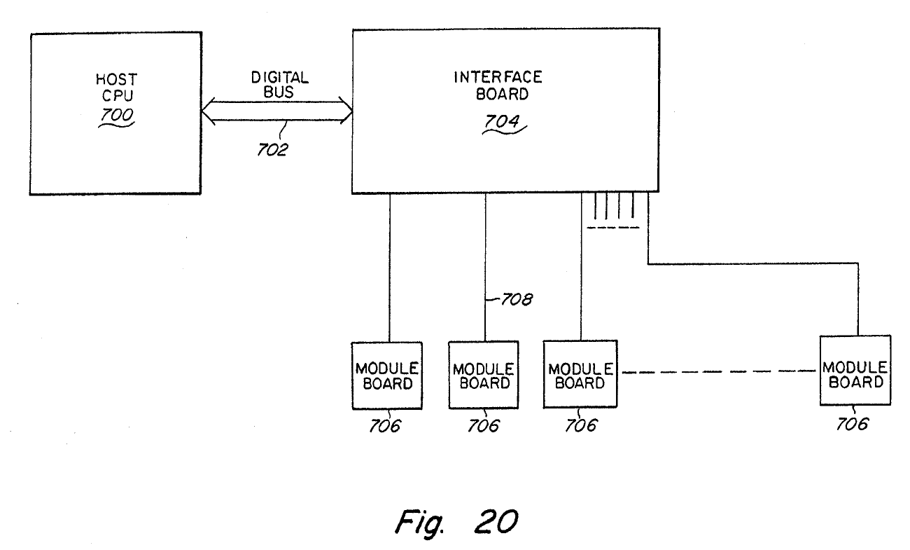

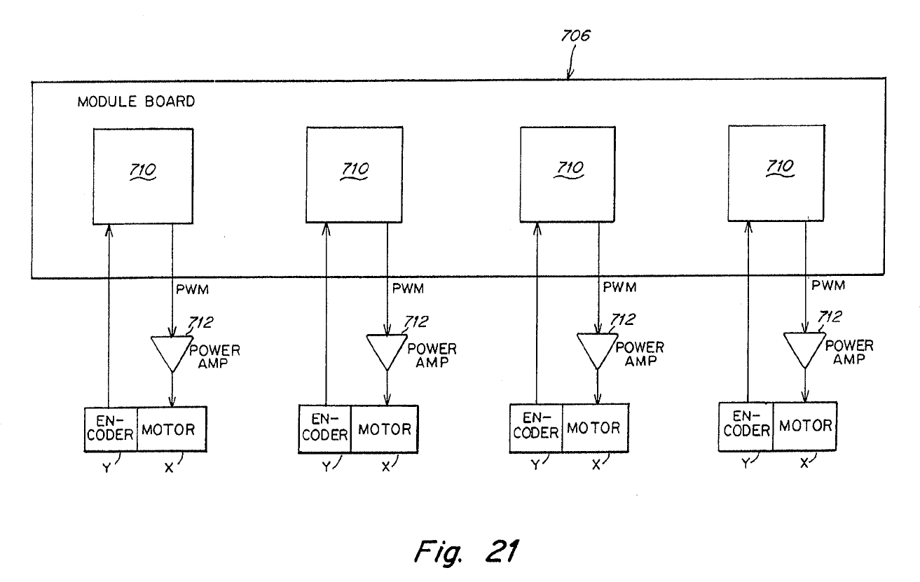

C5—Slave Guide Tube (FIGS. 19-19D)

Reference is now made to FIG. 19, a schematic diagram illustrating different placements of the guide tube 17. FIG. 19A illustrates left and right guide tubes substantially in the same position as illustrated in FIG. 1. For some surgical procedures, it may be advantageous to orient the tubes so that the curvatures are in the same direction. FIG. 19B shows the ends of the tubes pointing to the right, while FIG. 19C shows the ends of the tubes pointing to the left. Lastly, in FIG. 19D the ends of the tubes are shown converging but in a downwardly directed position. Regarding the different placements shown in FIG. 19, the adjustable clamp 25, illustrated in FIGS. 8A-8C may be useful, as this provides some added level of flexibility in supporting the positioning of the guide tubes on both the left and right side.

C6—Slave Motor Control (FIGS. 20-28)

FIGS. 20 and 21 are block diagrams of the motor control system of the present embodiment. In the system of FIG. 1, there are two instruments supported on either side of the operating table. Thus, there are in actuality two separate drive units 8. One of these is considered a left hand (LH) station and the other is considered a right hand (RH) station. Similarly, at the master station, on either side of the chair, as depicted in FIG. 1, there are left hand and right hand master station assemblies. Accordingly, there are a total of 28 (7×4) separate actions that are either sensed or controlled. This relates to seven separate degrees-of-motion at both the master and slave, as well as at left hand and right stations. In other embodiments there may be only a single station, such as either a left hand station or a right hand station. Also, other embodiments may employ fewer or greater numbers of degrees-of-motion as identified herein.

Regarding the master station side, there is at least one position encoder associated with each of degree-of-motion or degree-of-freedom. Also, as previously described, some of the described motions of the active joints have a combination of motor and encoder on a common shaft. With regard to the master station, all of the rotations represented by J1, J2 and J3 (see FIG. 2A) have associated therewith, not only encoders but also individual motors. At the hand assembly previously described, there are only encoders. However, the block diagram system of FIGS. 20 and 21 illustrates a combination with motor and encoder. If a motor is not used at a master station, then only the encoder signal is coupled to the system.

FIGS. 20 and 21 illustrate a multi-axis, high performance motor control system which may support anywhere from 8 to 64 axes, simultaneously, using either eight-bit parallel or pulse width modulated (PWM) signals. The motors themselves may be direct current, direct current brushless or stepper motors with a programmable digital filter/commutater. Each motor accommodates a standard incremental optical encoder.

The block diagram of FIG. 20 represents the basic components of the system. This includes a host computer 700, connected by a digital bus 702 to an interface board 704. The interface board 704 may be a conventional interface board for coupling signals between the digital bus and the eight individual module boards 706. The set of module boards is referred to as the motor control sub unit. Communication cables 708 intercouple the interface board 704 to eight separate module boards 706. The host computer 700 may be an Intel microprocessor based personal computer (PC) at a control station preferably running a Windows NT program communicating with the interface board 704 by way of a high-speed PCI bus 702 (5.0 KHz for eight channels to 700 Hz for 64 channels).

FIG. 21 shows one of the module boards 706. Each board 706 includes four motion control circuits 710. Each of the blocks 710 may be a Hewlett-Packard motion control integrated circuit. For example, each of these may be an IC identified as HCTL 1100. Also depicted in FIG. 21 is a power amplifier sub unit 712. The power amplifier sub unit is based on National Semiconductor's H-bridge power amplifier integrated circuits for providing PWM motor command signals. The power amplifier 712 associated with each of the blocks 710 couples to a motor X. Associated with motor X is encoder Y. Also note the connection back from each encoder to the block 710. In FIG. 21, although the connections are not specifically set forth, it is understood that signals intercouple between the block 710 and the interface board 704, as well as via bus 702 the host computer 700.

The motor control system may be implemented for example, in two ways. In a first method the user utilizes the motor control subunit 706 to effect four control modes: positional control, proportional velocity control, trapezoidal profile control and integral velocity control. Using any one of these modes means specifying desired positions or velocities for each motor, and the necessary control actions are computed by the motion control IC 710 of the motor control subunit, thereby greatly reducing the complexity of the control system software. However, in the case where none of the on-board control modes are appropriate for the application, the user may choose a second method in which a servo motor control software is implemented at the PC control station. Appropriate voltage signal outputs for each motor are computed by the PC control station and sent to the motor control/power amplifier unit (706, 712). Although the computation load is mostly placed on the control station's CPU in this case, there are available high performance computers and high speed PCI buses for data transfer which can accommodate this load.

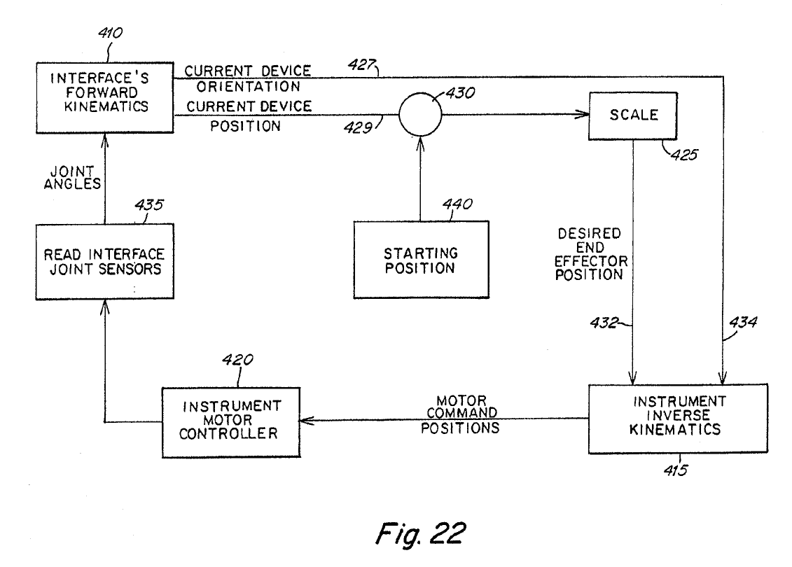

### D. Master-Slave Positioning and Orientation (FIGS. 22-28)

FIG. 22 provides an overview of control algorithm for the present embodiment. Its primary function is to move the instrument tool 18 in such a way that the motions of the instrument tool are precisely mapped to that of the surgeon interface device 3 in three dimensional space, thereby creating the feel of the tool being an extension of the surgeon's own hands. The control algorithm assumes that both the surgeon's input interface as well as the instrument system always start at predefined positions and orientations, and once the system is started, it repeats a series of steps at every sampling period. The predefined positions and orientations, relate to the initial positioning of the master and slave stations.

First, the joint sensors (box 435), which are optical encoders in the present embodiment, of the surgeon's interface system are read, and via forward kinematics (box 410) analysis, the current position (see line 429) and orientation (see line 427) of the input interface handle are determined. The translational motion of the surgeon's hand motion is scaled (box 425), whereas the orientation is not scaled, resulting in a desired position (see line 432) and orientation (see line 434) for the instrument tool. The results are then inputted into the inverse kinematics algorithms (box 415) for the instrument tool, and finally the necessary joint angles and insertion length of the instrument system are determined. The motor command positions are sent to the instrument motor controller (box 420) for commending the corresponding motors to positions such that the desired joint angles and insertion length are achieved.

With further reference to FIG. 22, it is noted that there is also provided an initial start position for the input device, indicated at box 440. The output of box 440 couples to a summation device 430. The output of device 430 couples to scale box 425. The initial handle (or hand assembly) position as indicated previously is established by first positioning of the handle at the master station so as to establish an initial master station handle orientation in three dimensional space. This is compared to the current handle position at device 430. This is then scaled by box 425 to provide the desired tool position on line 432 to the instrument inverse kinematics box 415.

The following is an analysis of the kinematic computations for both box 410 and box 415 in FIG. 22.

### Kinematic Computations

The present embodiment provides a surgeon with the feel of an instrument as being an extension of his own hand. The position and orientation of the instrument tool is mapped to that of the surgeon input interface device, and this mapping process is referred to as kinematic computations. The kinematic calculations can be divided into two sub-processes: forward kinematic computation of the surgeon user interface device, and inverse kinematic computation of the instrument tool.

### Forward Kinematic Computation

Based on the information provided by the joint angle sensors, which are optical encoders of the surgeon interface system, the forward kinematic computation determines the position and orientation of the handle in three dimensional space.

### 1. Position

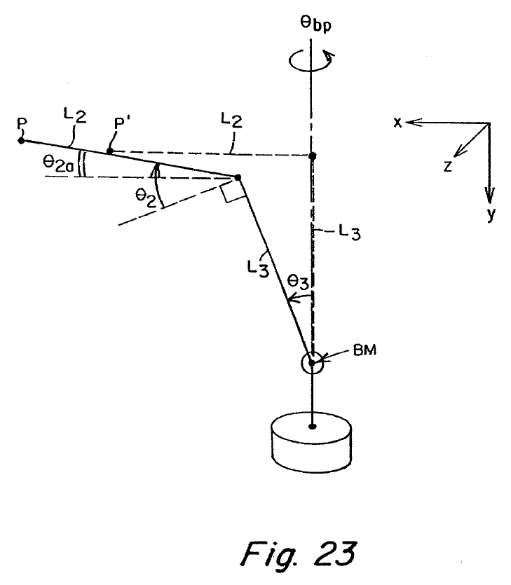

The position of the surgeon's wrist in three dimensional space is determined by simple geometric calculations. Referring to FIG. 23, the x, y, z directional positions of the wrist with respect to the reference coordinate are

Xₚ=(L₃ sin θ₃+L2 cos θ₂ₐ)cos θ_{bp}−L₂

Yₚ=−(L₃ cos θ₃+L2 sin θ₂ₐ)−L₃

Zₚ=(L₃ sin θ₃+L2 cos θ₂ₐ)sin θ_{bp}

where Xₚ, Yₚ, and Zₚ are wrist positions in the x, y, z directions, respectively.

These equations for Xₚ, Yₚ, and Zₚ represent respective magnitudes as measured from the initial reference coordination location, which is the location in FIG. 23 when θ₃ and θ₂ₐ are both zero degrees. This corresponds to the position wherein arm L2 is at right angles to arm L3 i.e., arm L2 is essentially horizontal and arm L3 is essentially vertical. That location is identified in FIG. 23 as coordinate location P′ where Xₚ=Yₚ=Zₚ=0. Deviations from this reference are calculated to determine the current position P.

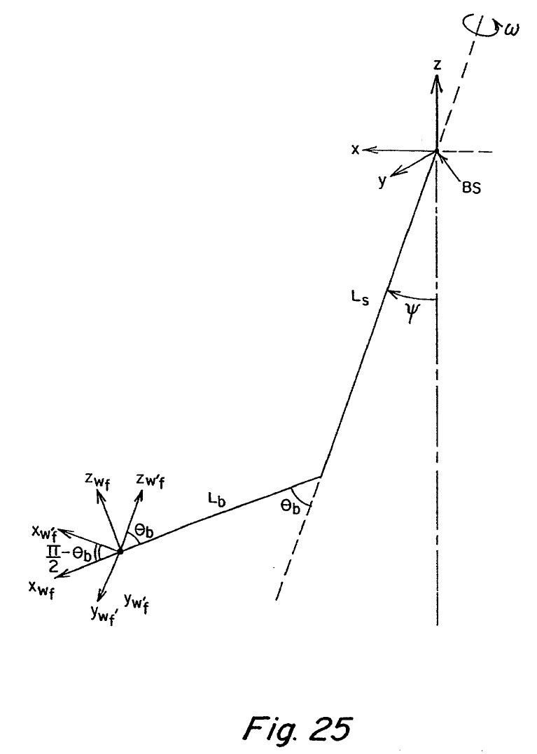

The reference coordinates for both the master and the slave are established with respect to a base location for each. In FIG. 23 it is location BM that corresponds structurally to the axis 60A in FIG. 2A. In FIG. 25 it is the location BS that corresponds structurally to the axis 225A in FIG. 2B. Because both the master and slave structures have predefined configurations when they are initialized, the locations of the master wrist 60A and the slave pivot 225A are known by the known dimensions of the respective structures. The predefined configuration of the master in the illustrated embodiment, per FIG. 23, relates to known lengths of arms L2 and L3, corresponding to arm 91 or arm 92, and arm 96 respectively. The predefined configuration of the slave is similarly defined, per FIG. 25, by dimensions of arms Lₛ and L_{b} and by initializing the slave unit with the guide tube 17 flat in one plane (dimension Y=O) and the arm Lₛ in line with the Z axis.

### 2. Orientation

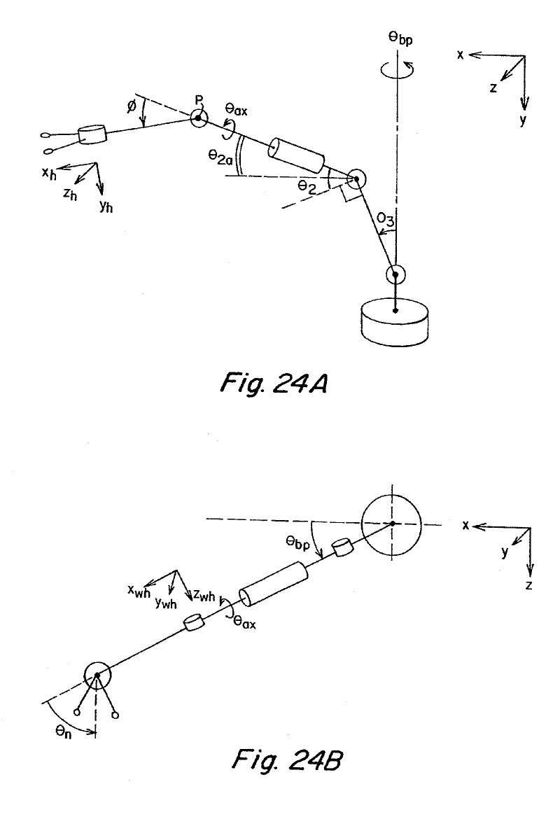

The orientation of the surgeon interface handle in three dimensional space is determined by a series of coordinate transformations for each joint angle. As shown in FIG. 24, the coordinate frame at the wrist joint is rotated with respect to the reference coordinate frame by joint movements θ_{bp}, θ₂, θ₃ and θₐₓ. Specifically, the wrist joint coordinate frame is rotated (−θ_{bp}) about the y axis, (−θ₂ₐ) about the z axis and θₐₓ about the x axis where θ₂ₐ is θ₂-θ₃. The resulting transformation matrix R_{wh} for the wrist joint coordinate frame with respect to the reference coordinate is then

$${R}_{wh} = [ \begin{matrix}{R}_{wh 11} & {R}_{wh 12} & {R}_{wh 13} \\ {R}_{wh 21} & {R}_{wh 22} & {R}_{wh 23} \\ {R}_{wh 31} & {R}_{wh 32} & {R}_{wh 33}\end{matrix} ]$$

Where R_{wh11}=cos θ_{bp1} cos θ₂ₐ

R_{wh12}=cos θ_{bp}1 sin θ₂ₐ cos θₐₓ−sin θ_{bp}1 sin θₐₓ

R_{wh13}=−cos θ_{bp1} sin θ₂ₐ sin θₐₓ−sin θ_{bp1} cos θₐₓ

R_{wh21}=−sin θ₂ₐ

R_{wh22}=cos θ₂ₐ cos θₐₓ

R_{wh23}=−cos θ₂ₐ sin θₐₓ

R_{wh31}=sin θ_{bp1} cos θ₂ₐ

R_{wh32}=sin θ_{bp} sin θ₂ₐ cos θₐₓ+cos θ_{bp1} sin θₐₓ

R_{wh33}=−sin θ_{bp1} sin θ₂ₐ sin θₐₓ+cos θ_{bp1} cos θₐₓ

Similarly, the handle coordinate frame rotates joint angles φ and θₕ) about the z and y axes with respect to the wrist coordinate frame. The transformation matrix R_{hwh} for handle coordinate frame with respect to the wrist coordinate is then

$${R}_{hwh} = [ \begin{matrix}{R}_{hwh 11} & {R}_{hwh 12} & {R}_{hwh 13} \\ {R}_{hwh 21} & {R}_{hwh 22} & {R}_{hwh 23} \\ {R}_{hwh 31} & {R}_{hwh 32} & {R}_{hwh 33}\end{matrix} ]$$

where R_{hwh11}=cos φ sin θₕ

R_{hwh11}=cos φ cos θₕ

R_{hwh12}=−sin φ

R_{hwh21}=sin φ cos θₕ

R_{hwh22}=cos φ

R_{hwh23}=−sin φ sin θₕ

R_{hwh31}=sin θₕ

R_{hwh32}=0

R_{hwh33}=cos θₕ

Therefore, the transformation matrix Rₕ for handle coordinate frame with respect to the reference coordinate is

Rₕ=R_{wh}R_{hwh}

### Inverse Kinematic Computation

Once the position and orientation of the surgeon interface handle are computed, the instrument tool is to be moved in such a way that the position of the tool's wrist joint in three dimensional space X_{w}, Y_{w}, Z_{w} with respect to the insertion point are proportional to the interface handle's positions by a scaling factor α.

(X_{w}−X_{w}_{_{—}}_{ref})=αXₚ

(Y_{w}−Y_{w}_{_{—}}_{ref})=αYₚ

(Z_{w}−Z_{w}_{_{—}}_{ref})=αZₚ

where X_{w}_{_{—}}_{ref}, Y_{w}_{_{—}}_{ref}, Z_{w}_{_{—}}_{ref} are the initial reference positions of the wrist joint. The orientations could be scaled as well, but in the current embodiment, are kept identical to that of the interface handle.

When X_{w}_{_{—}}_{ref},=Y_{w}_{_{—}}_{ref},=Z_{w}_{_{—}}_{ref}=0 the foregoing equations simplify to:

X_{w}=αXₚ

Y_{w}=αYₚ

Z_{w}=αZₚ

where (X_{w}, Y_{w}, Z_{w}), (Xₚ, Yₚ, Xₚ,) and α are the desired absolute position of the instrument, current position of the interface handle and scaling factor, respectively.

### 1. Position

The next task is to determine the joint angles ω, ψ and the insertion length Lₛ of the instrument, as shown in FIG. 25, necessary to achieve the desired positions of the tool's wrist joint. Given Y_{w}, the angle ω. is

$$ω = ( \frac{{Y}_{w}}{{L}_{bs}} ) ω = {sin}^{- 1} ( {Y}_{w} / {L}_{bs} ) or , \\ where {L}_{bs} = {L}_{b} sin {θ}_{b} .$$

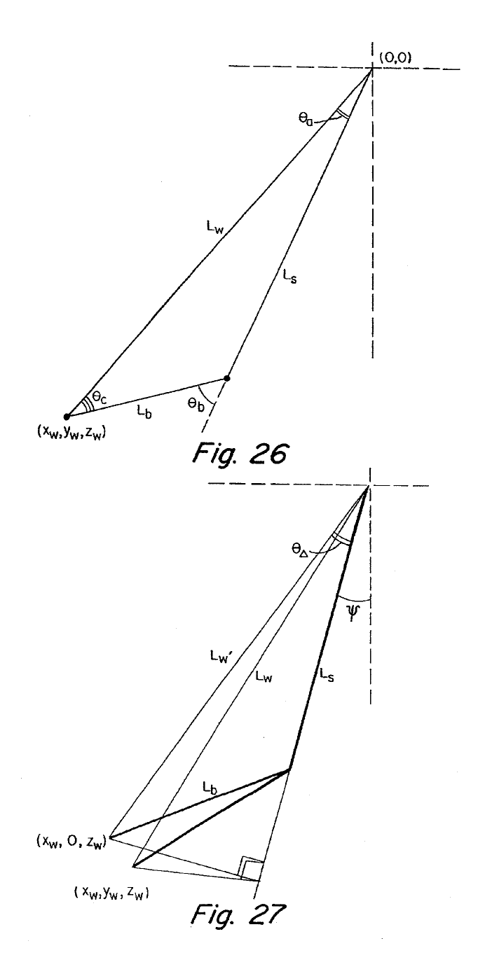

Referring to FIG. 26, the sine rule is used to determine the insertion length Lₛ of the instrument. Given the desired position of the tool's wrist joint, the distance from the insertion point to the wrist joint, L_{ws} is simply

L_{w}+√\vec{X_{w}²+Y_{w}²+Z_{w}²}

Then by the sine rule, the angle θₐ is

$${θ}_{a} = arcsin ( \frac{{L}_{b}}{{L}_{w}} sin {θ}_{b} ) , and {L}_{s} = {L}_{w} ( \frac{sin {θ}_{c}}{sin {θ}_{b}} ) where {θ}_{c} = {θ}_{b} - {θ}_{a}$$

Having determined ω and Lₛ, the last joint angle ψ can be found from the projection of the instrument on the x-z plane as shown in FIG. 27.

ψ=θ_{L′}_{_{w}}−θ_{Δ}

Where

$$\begin{matrix}{θ}_{Δ} = arccos ( \frac{{L}_{s} + {L}_{b} cos {θ}_{b}}{{L}_{w}^{′}} ) , \\ {θ}_{{L}_{w}^{′}} = arcsin ( \frac{{X}_{wo}}{{L}_{w}^{′}} ) ,\end{matrix}$$

$${L}_{w}^{′} = \sqrt{{X}_{w}^{2} + {Z}_{w}^{2}}$$

and X_{WO} is the x-axis wrist position in reference coordinate frame.

### 2. Orientation

The last step in kinematic computation for controlling the instrument is determining the appropriate joint angles of the tool such that its orientation is identical to that of the surgeon's interface handle. In other words, the transformation matrix of the tool must be identical to the transformation matrix of the interface handle, Rₕ

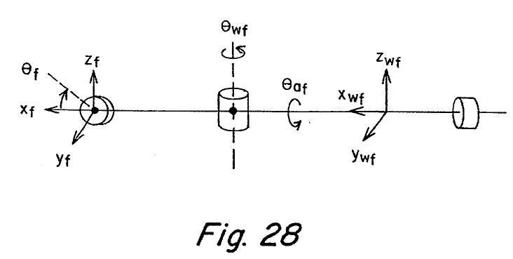

The orientation of the tool is determined by pitch (θ_{f}), yaw (θ_{wf}) and roll (θ_{af}) joint angles as well as the joint angles and ω and ψ as shown in FIG. 28. First, the starting coordinate is rotated (θ_{b}, −π/2) about the y-axis to be aligned with the reference coordinate, represented by the transformation matrix Rₒ

$${R}_{o} = [ \begin{matrix}sin {θ}_{b} & 0 & ( - cos {θ}_{b} ) \\ 0 & 1 & 0 \\ cos {θ}_{b} & 0 & sin {θ}_{b}\end{matrix} ]$$

The wrist joint coordinate is then rotated about the reference coordinate by angles (−ψ) about the y-axis and ω about the z-axis, resulting in the transformation matrix R_{w′f}.

$${Rw}^{′} f = [ \begin{matrix}cos ψcosω & ( - cos ψsinω ) & ( - sin ψ ) \\ sin ψ & cos ψ & 0 \\ sin ψcosω & ( - sin ψsinω ) & cos ψ\end{matrix} ]$$

followed by rotation of (π/2−θ_{b}) about the y-axis, represented by R_{wfw′f}.

$${R}_{{wf}^{′} wf} = [ \begin{matrix}sin {θ}_{b } & 0 & cos {θ}_{b} \\ 0 & 1 & 0 \\ - cos {θ}_{b} & 0 & sin {θ}_{b}\end{matrix} ]$$

which is equal to Rₜ^{o}.

Finally, the tool rolls (−θ_{af}) about the x-axis, yaws θ_{wf} about the z-axis and pitches (−θ_{f}) about the y-axis with respect to the wrist coordinate, are calculated resulting in transformation matrix R_{fwf}.

$${R}_{fwf} = [ \begin{matrix}{R}_{fwf 11} & {R}_{fwf 12} & {R}_{fwf 13} \\ {R}_{fwf 21} & {R}_{fwf 22} & {R}_{fwf 23} \\ {R}_{fwf 31} & {R}_{fwf 32} & {R}_{fwf 33}\end{matrix} ]$$

where R_{fwf11}=cos θ_{wf} cos θ_{f}

R_{fwf12}=−sin θ_{wf}

R_{fwf13}=−cos θ_{wf} sin θ_{f}

R_{fwf21}=cOS θ_{af} sin θ_{wf} cos θ_{f}+sin θ_{af} sin θ_{f}

R_{fwf22}=cos θ_{af} cos θ_{wf}

R_{fwf23}=−cos θ_{af} sin θ_{wf} sin θ_{f}+sin θ_{af} cos θ_{f}

R_{fwf31}=−sin θ_{af} sin θ_{wf} cos θ_{f}+cos θ_{af} sin θ_{f}

R_{fwf32}=−sin θ_{af} cos θ_{wf}

R_{fwf33}=sin θ_{af} sin θ_{wf} sin θ_{f}+cos θ_{af} cos θ_{f}

Therefore the transformation matrix of the tool R_{f} with respect to the original coordinate is R_{f}=RₒR_{wf′}Rₒ^{T}R_{fwf}.

Since R_{f} is identical to Rₕ of the interface handle, R_{w} can be defined by

R_{fwf}=RₒR_{wf′}^{T}Rₒ^{T}Rₕ=R_{c}

$${R}_{fwf} = {R}_{o} {R}_{wf}^{t} , {R}_{o}^{t} {R}_{h} = {R}_{c} = [ \begin{matrix}{R}_{c 11} & {R}_{c 12} & {R}_{c 13} \\ {R}_{c 21} & {R}_{c 22} & {R}_{c 23} \\ {R}_{c 31} & {R}_{c 32} & {R}_{c 33}\end{matrix} ]$$

where the matrix R_{c} can be fully computed with known values. Using the computed values of R_{c} and comparing to the elements of R_{fwf}, we can finally determine the necessary joint angles of the tool.

$$\begin{matrix}{θ}_{wf} = arcsin ( - {R}_{c 12} ) , \\ {θ}_{f} = arccos ( \frac{{R}_{c 11}}{cos {θ}_{wf}} ) = arcsin ( \frac{- {R}_{c 13}}{cos {θ}_{wf}} ) \\ {θ}_{af} = arccos ( \frac{{R}_{c 22}}{cos {θ}_{f}} ) = arcsin ( \frac{- {R}_{c 32}}{cos {θ}_{wf}} )\end{matrix}$$

The actuators, which are motors in the current embodiment, are then instructed to move to positions such that the determined joint angles and insertion length are achieved.

Now reference is made to the following algorithm that is used in association with the system of the present invention. First are presented certain definitions.

### Variable Definitions

### RH-Right Hand, LH-Left Hand

s_Ls_RH Linear slider joint for RH slave

s_Xi_RH Lateral motion joint for RH slave (big disk in front of slider)

s_Omega_RH Up/down motion joint for RH slave (rotates curved tube)

s_Axl_RH Axial rotation joint for RH slave (rotates instrument insert along its axis)

s_f1_RH Finger 1 for RH slave

s_f2_RH Finger 2 for RH slave

s_wrist_RH Wrist joint for RH slave

m_base_RH Base rotation joint for RH master

m_shoulder_RH Shoulder joint for RH master

m_elbow_RH Elbow joint for RH master

m_Axl_RH Axial rotation joint for RH master

m_f1_RH Finger 1 for RH master

m_f2_RH Finger 2 for RH master

m_wrist_RH Wrist joint for RH master

Radian[i] Motor axle angle for joint no. i with i being one of above joints

Des_Rad[i] Desired motor axle angle for joint no. i

Des_Vel[i] Desired motor axle angular velocity for joint no. i

Mout_f[i] Motor command output for joint no. i

Thetabp1_m_RH Angle of base rotation joint for RH master

Theta2_m_RH Angle of elbow joint for RH master

Theta3_m_RH Angle of shoulder joint for RH master

Xw_m_RH Position of RH master handle in X-axis

Yw_m_RH Position of RH master handle in Y-axis

Zw_m RH Position of RH master handle in Z-axis

Xwref_m_RH Reference position of RH master handle in X-axis

Ywref_m_RH Reference position of RH master handle in Y-axis

Zwref_m_RH Reference position of RH master handle in Z-axis

Phi_f_m_RH Angle of wrist joint for RH master

Theta_f1_m_RH Angle of finger 1 for RH master

Theta_f2_m_RH Angle of finger 2 for RH master

ThetaAxl_m_RH Angle of axial rotation joint for RH master

Theta_h_m_RH Angle of mid line of fingers for RH master

Theta_f_m_RH Angle of fingers from the mid line for RH master

Xw_s_RH Position of RH slave in X-axis

Yw_s_RH Position of RH slave in Y-axis

Zw_s_RH Position of RH slave in Z-axis

Xwref_s_RH Reference position of RH slave in X-axis

Ywref_s_RH Reference position of RH slave in Y-axis

Zwref_s_RH Reference position of RH slave in Z-axis

alpha Master-to-slave motion scaling factor

Xw_s_b1_RH Motion boundary 1 of RH slave in X-axis

Xw_s_b2_RH Motion boundary 2 of RH slave in X-axis

Yw_s_b1_RH Motion boundary 1 of RH slave in Y-axis

Yw_s_b2_RH Motion boundary 2 of RH slave in Y-axis

Note the motion boundaries of the slave are used to define the virtual boundaries for the master system, and do not directly impose boundaries on the slave system.

The following represents the steps through which the algorithm proceeds.

1. The system is started, and the position encoders are initialized to zero. This ASSUMES that the system started in predefined configuration.

```
/* Preset Encoder Position for all axis */
for(i=0; i<32; ++i)
{
                    SetEncoder[i]=0;
}
/* Convert encoder count to radian */
for(i=0;i<32;i++)
{
                    Radian[i] = Enc_to_Rad(Encoder[i]);
}
```

2. Bring the system to operating positions, Des_Rad[i], and hold the positions until the operator hits the keyboard, in which case the program proceeds to next step.

```
While(!kbhit( ))
{
        for(i=0;i<14;i++)  /* compute motorout for slave robots*/
        {
            Des_Vel[i] = 0.0;
            Err_Rad[i] = Des_Rad[i] − Radian[i];
            Err_Vel[i] = Des_Vel[i] − Velocity[i];
            kpcmd = Kp[i]*Err_Rad[i];
            kdcmd = (Kp[i]*Td[i])*Err_Vel[i];
            Mout_f[i] = kpcmd + kdcmd; /* Command output
            to motor */
        }
        for(i=14;i<28;i++)  /* compute motorout for master robot */
        {
            Des_Vel[i] = 0.0;
            Err_Rad[i] = Des_Rad[i] − Radian[i];
            Err_Vel[i] = Des_Vel[i] − Velocity[i];
            kpcmd = Kp[i]*Err_Rad[i];
            kdcmd = (Kp[i]*Td[i])*Err_Vel[i];
            Mout_f[i] = kpcmd + kdcmd; /* Command output
            to motor */
        }
}
```

3. Based on the assumption that the system started at the predefined configuration, the forward kinematic computations are performed respectively for the master and the slave systems to find the initial positions/orientations of handles/tools.

```
        /* Compute Initial Positions of Wrist for Right Hand Master */
        Thetabp1o_m_RH =−Radian[m_base_RH]/PR_bp1;
        Theta3o_m_RH=−Radian[m_shoulder_RH]/PR_3;
        Thetabp1_m_RH=Thetabp1o_m_RH;
        Theta3_m_RH=Theta3o_m_RH;
        Theta2o_m_RH =Theta3o_m_RH−
        Radian[m_elbow_RH]/PR_2;
        Theta2_m_RH=Theta2o_m_RH;
        Theta2A_m_RH=(Theta2_m_RH−Theta3_m_RH);
        Theta2A_eff_m_RH=Theta2A_m_RH+Theta_OS_m;
        L_m_RH=(L3_m*sin(Theta3o_m_RH)+
        L2_eff_m*cos(Theta2A
        eff_m_RH));
        Xwo_m_RH=L_m_RH*cos(Thetabp1o_m_RH);
        Ywo_m_RH=−
        (L3_m +L2_eff_m*sin(Theta2A_eff_m_RH));
        Zwo_m_RH=L_m_RH*sin(Thetabp1o_m_RH);
        /* Set these initial positions as the reference positions. */
        Xwref_m_RH=Xwo_m_RH=L2 (Fig. 23)
        Ywref_m_RH=Ywo_m_RH=L3
        Zwref_m_RH=Zwo_m_RH=0
        /* Initial Position of the Wrist for Right Hand Slave based
on predefined configurations */
        Ls_RH=Ls;
        Xwo_s_RH=Lbs=Xref_s_RH
        Ywo_s_RH=0.0=Yref_s_RH
        Zwo_s_RH=−(Ls_RH+Lbc)=Zref_s_RH
        /* Compute Initial Orientations for Right Hand Handle */
        Phi_f_m_RH=Radian[m_wrist_RH];
        Theta_f1_m_RH=Radian[m_f1_RH];
        Theta_f2_m_RH=−Radian[m_f2_RH]−Theta_f1_m_RH;
        ThetaAxl_m_RH=Radian[m_Axl_RH];
        Theta_h_m_RH=(Theta_f1_m_RH−Theta_f2_m_RH)/
2.0; /* angle of midline
        Theta_f_m_RH=(Theta_f1_m_RH+Theta_f2_m_RH)/
2.0;/* angle of fingers from mid line */
        /* Repeat for Left Hand Handle and Slave Instrument */
```

4. Repeat the procedure of computing initial positions/orientations of handle and tool of left hand based on predefined configurations.

5. Read starting time.

```
        /* Read starting time: init_time */
        QueryPerformanceCounter(&hirescount);
        dCounter=(double)hirescount.LowPart+
(double)hirescount.HighPart*(double)(− 4294967296);
        QueryPerformanceFrequency(&freq);
        init_time=(double)(dCounter/freq.LowPart);
        prev_time=0.0;
```

6. Read encoder values of master/slave system, and current time.

```
/* Read encoder counters */
for(i=1; i<9;++i)
{
    Read_Encoder(i);
}
/* Convert encoder counts to radian */
for(i=0;i<32;i++)
{
    Radian[i] = Enc_to_Rad(Encoder[i]);
}
/* Get current time */
QueryPerformanceCounter(&hirescount);
dCounter = (double)hirescount.LowPart + (double)hirescount.HighPart *
(double)(4294967296);
time_now = (double)(dCounter/freq.LowPart) − init_time;
delta_time3 = delta_time2;
delta_time2 = delta_time1;
delta_time1 = time_now − prev_time;
prev_time = time_now;
```

7. Compute current positions/orientations of master handle for Right Hand.

```
        /* Compute master handle's position for right hand */
        Thetabp1_m_RH=−Radian[m_base_RH]/PR_bp1;
        Theta3_m_RH=−Radian[m_shoulder_RH]/PR_3;
        Theta2_m_RH=Theta3_m_RH−Radian[m_elbow_RH]/
        PR_2;
        Theta2A_m_RH=(Theta2_m_RH−Theta3_m_RH);
        Theta2A_eff_m_RH=Theta2A_m_RH+Theta_OS_m;
        L_m_RH=(L3_m*sin_Theta3_m+L2_eff_m*
        cos_Theta2A_eff_m_);
        Xw_m_RH=L_m_RH*cos_Thetabp1_m;
        Yw_m_RH=−(L3_m*cos_Theta3_m+L2_eff_m*
        sin_Theta2A_eff_m);
        Zw_m_RH=L_m_RH*sin_Thetabp1_m;
        /* Compute master handle's orientation for right hand */
        Phi_f_m_RH=Radian[m_wrist_RH];
        Theta_f1_m_RH=Radian[m_f1_RH];
        Theta_f2_m_RH=−Radian[m_f2_RH]−Theta_f1_m_RH;
        ThetaAxl_m_RH=Radian[m_Axl_RH];
        Theta_h_m_RH=(Theta_f1_m_RH−Theta_f2_m_RH)/
2.0; /* angle of midline */
        Theta_f_m_RH=(Theta_f1_m_RH+Theta_f2_m RH)/
2.0; /* angle of fingers from mid line */
        /* Perform coordinate transformation to handle's coordinate */
        Rwh11=cos_Thetabp1_m*cos_Theta2A_m;
        Rwh12=−sin_Thetabp1_m*sin_ThetaAxl_m+
        cos_Thetabp1_m*sin_Theta2A_m*cos_ThetaAxl_m;
        Rwh13=−sin_Thetabp1_m*cos_ThetaAxl_m−
        cos_Thetabp1_m*sin_Theta2A_m*sin_ThetaAxl_m;
        Rwh21=−sin_Theta2A_m;
        Rwh22=cos_Theta2A_m*cos_ThetaAxl_m;
        Rwh23=−cos_Theta2A_m*sin_ThetaAxl_m;
        Rwh31=sin_Thetabp1_m*cos_Theta2A_m;
        Rwh32=cos_Thetabp1_m*sin_ThetaAxl_m+
        sin_Thetabp1_m*sin_Theta2A_m*cos_ThetaAxl_m;
        Rwh33=cos_Thetabp1_m*cos_ThetaAxl_m−
        sin_Thetabp1_m*sin_Theta2A_m*sin_ThetaAxl_m;
        Rhr11=cos_Phi_f_m*cos_Theta_h_m;
        Rhr12=−sin_Phi_f_m;
        Rhr13=−cos_Phi_f_m*sin_Theta_h_m;
        Rhr21=sin_Phi_f_m*cos_Theta_h_m;
        Rhr22=cos_Phi_f_m;
        Rhr23=−sin_Phi_f_m*sin_Theta_h_m;
        Rhr31=sin_Theta_h_m;
        Rhr32 =0.0;
        Rhr33=cos_Theta_h_m;
        Rh11=Rwh11*Rhr11+Rwh12*Rhr21+Rwh13*Rhr31;
        Rh12=Rwh11*Rhr12+Rwh12*Rhr22+Rwh13*Rhr32;
        Rh13=Rwh11*Rhr13+Rwh12*Rhr23+Rwh13*Rhr33;
        Rh21=Rwh21*Rhr11+Rwh22*Rhr21+Rwh23*Rhr31;
        Rh22=Rwh21*Rhr12+Rwh22*Rhr22+Rwh23*Rhr32;
        Rh23=Rwh21*Rhr13+Rwh22*Rhr23+Rwh23*Rhr33;
        Rh31=Rwh31*Rhr11+Rwh32*Rhr21+Rwh33*Rhr31;
        Rh32=Rwh31*Rhr12+Rwh32*Rhr22+Rwh33*Rhr32;
        Rh33=Rwh31*Rhr13+Rwh32*Rhr23+Rwh33*Rhr33;
```

8. Desired tool position is computed for right hand.

/* Movement of master handle is scaled by alpha for tool position */
Xw_s_RH=alpha*(Xw_m_RH−Xwref_m_RH)+Xwref_s_RH;
Yw_s_RH=alpha*(Yw_m_RH−Ywref_m_RH)+Ywref_s_RH;
Zw_s_RH=alpha*(Zw_m_RH−Zwref_m_RH)+Zwref_s_RH;

/* The next step is to perform a coordinate transformation from the wrist coordinate (refer to FIG. 25 and coordinate Xwf, Ywf and Zwf) to a coordinate aligned with the tube arm Ls. This is basically a fixed 450 transformation (refer in FIG. 25 to θ_{b}) involving the sin and cos of θ_{b} as expressed below. */

Xwo_s._RH=Xw_s_RH*sin_Theta_b+Zw_s_RH*cos_Theta_b;
Ywo_s_RH=Yw_s_RH;
Zwo_s_RH=−Xw_s_RH*cos_Theta_b+Zw_s_RH*sin_Theta_b;

9. Perform inverse kinematic computation for the right hand to obtain necessary joint angles of the slave system such that tool position/orientation matches that of master handle.

Omega_RH=a sin(Ywo_s_RH/Lbs);
Lw=sqrt(pow(Xwo_s_RH,2)+pow(Ywo_s_RH,2)+
pow(Zwo_s_RH,2));
Theta_a=a sin(Lb/Lw*sin_Theta_b);
Theta_c=Theta_b−Theta_a;
Ls_RH=Lw*(sin(Theta_c)/sin_Theta_b);
Lwp=sqrt(pow(Lw,2)−pow(Ywo_s_RH,2));
Theta_Lwp=a sin(Xwo_s_RH/Lwp);
Xi_RH=Theta_Lwp−Theta_delta;
sin_Omega=sin(Omega_RH);
cos_Omega=cos(Omega_RH);
sin_Xi=sin(Xi_RH);
cos_Xi=cos(Xi_RH);
Ra11=cos_Xi*cos_Omega*sin_Theta_b+sin_Xi*cos_Theta_b;
Ra12=sin_Omega*sin_Theta_b;
Ra13=sin_Xi*cos_Omega*sin_Theta_b−cos_Xi*cos_Theta_b;
Ra21=−cos_Xi*sin_Omega;
Ra22=cos_Omega;
Ra23=−sin_Xi*sin_Omega;
Ra31=cos_Xi*cos_Omega*cos_Theta_b−sin_Xi*sin_Theta_b;
Ra32=sin_Omega*cos_Theta_b;
Ra33=sin_Xi*cos_Omega*cos_Theta_b+cos_Xi*sin_Theta_b;
Rb11=Ra11*sin_Theta_b−Ra13*cos_Theta_b;
Rb12=Ra12;
Rb13=Ra11*cos_Theta_b+Ra13*sin_Theta_b;
Rb21=Ra21*sin_Theta_b−Ra23*cos_Theta_b;
Rb22=Ra22;
Rb23=Ra21*cos_Theta_b+Ra23*sin_Theta_b;
Rb31=Ra31*sin_Theta_b−Ra33*cos_Theta_b;
Rb32=Ra32;
Rb33=Ra31*cos_Theta_b+Ra33*sin_Theta_b;
Rc11=Rb11*Rh11+Rb12*Rh21+Rb13*Rh31;
Rc12=Rb11*Rh12+Rb12*Rh22+Rb13*Rh32;
Rc13=Rb11*Rh13+Rb12*Rh23+Rb13*Rh33;
Rc21=Rb21*Rh11+Rb22*Rh21+Rb23*Rh31;
Rc22=Rb21*Rh12+Rb22*Rh22+Rb23*Rh32;
Rc23=Rb21*Rh13+Rb22*Rh23+Rb23*Rh33;
Rc31=Rb31*Rh11+Rb32*Rh21+Rb33*Rh31;
Rc32=Rb31*Rh12+Rb32*Rh22+Rb33*Rh32;
Rc33=Rb31*Rh13+Rb32*Rh23+Rb33*Rh33;
sin_Theta_wf_s=−Rc12;
Theta_wf_s_RH=a sin(sin_Theta_wf_s);
cos_Theta_wf_s=cos(Theta_wf_s_RH);
/* Compute Theta_f_s_RH */
var1=Rc11/cos_Theta_wf_s;
var2=−Rc13/cos_Theta_wf_s;
Theta_f_s_RH=a sin(var2) or a cos(var1) depending or region;
/* Compute ThetaAxl_s_RH */
var1=Rc22/cos_Theta_wf_s;
var2=−Rc32/cos_Theta_wf_s;
ThetaAxl_s_RH=a sin(var2) or a cos(var1) depending or region;

10. Repeat steps 7-9 for left hand system.

11. Determine motor axle angles necessary to achieve desired positions/orientations of the slave systems, and command the motors to the determined positions.

```
        Des_Rad[s_Ls_RH]=63.04*(Ls_RH−Ls_init_RH−0.75*(Xi_RH−Xi_init_RH));
        Des_Rad[s_Xi_RH]=−126.08*(Xi_RH−Xi_init_RH);
        Des_Rad[s_Omega_RH]=−23.64*(Omega_RH−Omega_init_RH);
        Des_Rad[s_Axl_RH]=−23.64*1.3333*(ThetaAxl_s_RH+Omega_RH−
        Omega_init_RH);
        Des_Rad[s_wrist_RH]=18.9*Theta_wf_s_RH;
        Des_Rad[s_f1_RH]=18.9*(Theta_f_s_RH+Theta_f_m_RH);
        Des_Rad[s_f2_RH]=18.9*(−Theta_f_s_RH+Theta_f_m_RH);
        Des_Rad[s_Ls_LH]=−63.04*(Ls_LH−Ls_init_LH−0.75*(Xi_LH−Xi_init_LH));
        Des_Rad[s_Xi_LH]=126.08*(Xi_LH−Xi_init_LH);
        Des_Rad[s_Omega_LH]=−23.64*(Omega_LH−Omega_init_LH);
        Des_Rad[s_Axl_LH]=−23.64*1.3333*(ThetaAxl_s_LH+Omega_LH−
Omega_init_LH);
        Des_Rad[s_wrist_LH]=18.9*Theta_wf_s_LH;
        Des_Rad[s_f1_LH]=−18.9*(−Theta_f_s_LH−Theta_f_m_LH);
        Des_Rad[s_f2_LH]=−18.9*(Theta_f_s_LH−Theta_f_m_LH);
        /* Compute motor output for slave systems */
        for(i=0;i<14;i++)
        {
            Des_Vel[i]=0.0;
            Err_Rad[i]=Des_Rad[i]−Radian[i];
            Err_Vel[i]=Des_Vel[i]−Velocity[i];
            kpcmd=Kp[i]*ErrRad[i];
            kdcmd=(Kp[i]*Td[i])*Err_Vel[i];
            Mout_f[i]=kpcmd+kdcmd;
        }
        /* Virtual boundaries for master handles */
        if (Xwo_s_RH>=Xw_s_b1_RH)
        {
            Fx_RH=3.0*k_master*(Xwo_s_RH−Xw_s_b1_RH);
            Mout_f[m_base_RH]=Fx_RH*cos(0.7854
(Radian[m_base_RH]/14.8));
            Mout_f[m_shoulder_RH]=Fx_RH*sin(0.7854−(Radian[m_base
RH]/14.8))−1.0*Radian[m_shoulder_RH];
        }
        else if (Xwo_s_RH<=Xw_s_b2_RH)
        {
            Fx_RH=k_master*(Xwo_s_RH−Xw_s_b2_RH);
            Mout_f[m_base_RH]=Fx_RH*cos(0.7854−
(Radian[m_base_RH]/14.8));
            Mout_f[m_shoulder_RH]=Fx_RH*sin(0.7854−
(Radian[m_base_RH]/14.8))−1.0*Radi- an[m_shoulder_RH];
        }
        else
        {
            Mout_f[m_base_RH]=0.0;
            Mout_f[m_shoulder_RH]=−1.0*Radian[m_shoulder_RH];
        }
        if (Ywo_s_RH>=Yw_s_b2_RH)
        {
            Mout_f[m_elbow_RH]=−k_master*(Ywo_s_RH−Yw_s_b2_RH);
        }
        else if(Ywo_s_RH<=Yw_s_b1_RH)
        {
            Mout_f[m_elbow_RH]=−k_master*(Ywo_s_RH−Yw_s_b1_RH);
        }
        else Mout_f[m_elbow RH]=0.0;
        /*Repeat for left master handle */
```

12. Go back to step 6 and repeat.

Previously there has been described an algorithm for providing controlled operation between the master and slave units. The following description relates this operation to the system of FIGS. 1-2.

The controller 9 receives input signals from the input device 3 that represent the relative positions of the different portions of the input device. These relative positions are then used to drive the instrument 14 to a corresponding set of relative positions. For example, the input 96 is rotatably connected to the first link at an elbow joint 94. Connected to the second link 96 opposite the elbow joint 94 is a wrist joint 98A and two fingers. A surgeon may attach a thumb and forefinger to the two fingers and move the input device to drive the instrument 14.

As the surgeon operates the input device, rotational position of the base (Thetabp1_m_RH), the rotational position of the first link relative to the base (Theta3_m_RH), the rotational position of the second link relative to the first link (Theta2_m_RH), the angle of the wrist joint relative to the second link (PHI_f_m_RH, i.e., the angle the wrist joint is rotated about an axis perpendicular to the length of the second link), the rotary angle of the wrist joint relative to the second link (ThetaAxl_m_RH, i.e., the angle the wrist joint is rotate about an axis parallel to the length of the second link), and the angles of the fingers (Theta_f1_m_RH and Theta_f2_m_RH) are provided to the controller.

When the surgical instrument is first started, the controller initializes all of the position encoders in the instrument 14 and the input device 3, assuming that the system has been started in a desired initial configuration. See Sections 1-3 of the algorithm. The initial position of the input device, e.g., Xwo_m_RH, Ywo_m_RH, and Zwo_m_RH, is then used to establish a reference position for the input device, Xwref_m_RH, Ywref_m_RH, and Zwref_m_RH. See Section 3 of the algorithm. Initial positions are also established for the instrument 14 based on the dimensions of the instrument 14. See Section 3 of the algorithm.

With reference to Section 3 of the algorithm, it is noted that there is an assignment of the initial position of the wrist for the slave, and that this is not a forward kinematics calculation based upon joint angles, but rather is a number based upon the predefined configuration of the slave unit. The coordinate of the slave relates to fixed physical dimensions of the instrument As the surgeon moves the input device 3, the encoder values for the input device are read and used to compute the current absolute position of the input device, i.e., Xw_m_RH Yw_m_RH, and Zw_m_RH. See Sections 6 and 7 of the algorithm. The controller then determines the desired position of the tool 18 (Xw_s_RH, Yw_s_RH and Zw_s_RH) based on the current position of the input device (Xw_m_RH Yw_m_RH, and Zw_m_RH), the reference position for the input device (Xwref_m_RH, Ywref_m_RH, and Zwref_m RH) and the reference position for the instrument 14 (Xwref_s_RH, Ywref_s_RH, and Zwref_s_RH). See Section 8 of the algorithm. The desired position of the tool 18 (Xw_s_RH, Yw_s_RH, and Zw_s_RH) is then transformed by a 45.degree. coordinate transformation giving the desired position (Xwo_s_RH, Ywo_s_RH, Zwo_s_RH) which is used to determine joint angles and drive motor angles for the instrument 14 orientation to match that of the input device. See Sections 8-11 of the algorithm. Thus, movement of the surgical instrument 14 is determined based on the current absolute position of the input device, as well as the initial positions of the input device and the instrument at the time of system start-up.

### E. Select Features of Described Embodiment

The control in accordance with the present embodiment, as exemplified by the foregoing description and algorithm, provides an improvement in structure and operation while operating in a relatively simple manner. For example, the control employs a technique whereby the absolute position of the surgeon input device is translated into control signals to move the instrument to a corresponding absolute position. This technique is possible at least in part because of the particular construction of the instrument and controllable instrument holder, which essentially replace the cumbersome prior art multi-arm structures including one or more passive joints. Here there is initialized an all active joint construction, including primarily only a single instrument holder having a well-defined configuration with respect to the inserted instrument.

Some prior-art systems rely upon passive joints to initially position the distal tip of the surgical instrument. Because the positions of the passive joints are initially unknown, the position of the distal tip of the surgical instrument with respect to the robot (instrument holder) is also unknown. Therefore, these systems require an initial calculation procedure. This involves the reading of joint angles and the computation of the forward kinematics of all elements constituting the slave. This step is necessary because the joint positions of the slave are essentially unknown at the beginning of the procedure.

On the other hand, in accordance with the present invention it is not necessary to read an initial position of joint angles in order to determine an initial position of the distal tip of the surgical instrument. The system of the present invention, which preferably employs no passive joints, has the initial position of the distal tip of the surgical instrument known with respect to the base of the instrument. The instrument is constructed with known dimensions, such as between base pivot 225 and the wrist (303 at axis 306 in FIG. 2D) of the tool 18. Further, the instrument is initially inserted by the surgeon in a known configuration, such as illustrated in FIGS. 9 and 10, where the dimensions and orientations of the instrument insert and adaptor guide tube are known with respect to the base (pivot 225). Therefore, an initial position of the surgical instrument distal tip need not be calculated before the system is used.

The system of the present embodiment is fixed to the end of a static mount (bracket 25 on post 19) which is manually maneuvered over the patient, such as illustrated in FIG. 1. since the initial position of the surgical instrument tip (tool 18) with respect to the base (pivot 225) of the articulate mechanism is invariant, the joint positions are neither read nor is the forward kinematics computed during the initial setup. Thus, the initial position of the surgical instrument tip is neither computed nor calculated. In addition, because the base of the system in accordance with the present embodiment is not necessarily fixed directly to the surgical table, but rather movable during a surgical procedure, the initial position of the surgical instrument in a world coordinate system is not knowable.

Another advantage of the present system is that the instrument does not use the incision in the patient to define a pivot point of the instrument. Rather, the pivot point of the instrument is defined by the kinematics of the mechanism, independent of the patient incision, the patient himself, or the procedure. Actually, the pivot point in the present system is defined even before the instrument enters the patient, because it is a pivot point of the instrument itself. This arrangement limits trauma to the patient in an area around the incision.

From an illustrative standpoint, the base of the instrument may be considered as pivot 225 (FIG. 8), and the wrist may be the pivot location 604 (axis) depicted in FIG. 16B (or axis 306 in FIG. 2D). The guide tube 17 has known dimensions and because there are no other joints (active or passive) between the pivot 225 and wrist joint, all of the intervening dimensions are known. Also, the instrument when placed in position has a predefined configuration such as that illustrated in FIGS. 1, 9 and 10 with the guide tube flat is one plane.

The guide tube 17 may also have an alignment mark therealong essentially in line with the pivot 225, as shown in FIG. 9. This marks the location where the guide tube 17 is at the patient incision point. The result is minimal trauma to the patient occasioned by any pivoting action about pivot 225.

Another advantage is the decoupling nature of the present system. This decoupling enables the slave unit to be readily portable. Here the instrument, drive unit and controller are decouplable. A sterilized adaptor 15 is inserted into a patient, then coupled to a non-sterile drive unit 8 (outside the sterile field). Instrument inserts 16 are then removably attached to the surgical adaptor to perform the surgical procedure. The system of the present embodiment separates the drive unit 8 from the instruments 16. In this way, the instruments can be maintained as sterile, but the drive unit need not be sterilized. Furthermore, at the time of insertion, the adaptor 15 is preferably decoupled from the drive unit 8 so it can be readily manually maneuvered to achieve the proper position of the instrument relative to the patient and the patient's incision.

In accordance with the present embodiment, the instrument inserts 16 are not connected to the controller 9 by way of any input/output port configuration. Rather, the present system employs an exclusively mechanical arrangement that is effected remotely and includes mechanical cables and flexible conduits coupling to a remote motor drive unit 8. This provides the advantage that the instrument is purely mechanical and does not need to be contained within a sterile barrier. The instrument may be autoclaved, gas sterilized or disposed in total or in part.

The present system also provides an instrument that is far less complex than prior art robotic system. The instrument is far smaller than that of a typical prior art robotic system, because the actuators (motors) are not housed in the articulate structure in the present system. Because the actuators are remote, they may be placed under the operating table or in another convenient location and out of the sterile field. Because the drive unit is fixed and stationary, the motors may be of arbitrary size and configuration, without effecting the articulated mechanics. Finally, the design allows multiple, specialized instruments to be coupled to the remote motors. This allows one to design an instrument for particular surgical disciplines including, but not limited to, such disciplines as cardiac, spinal, thoracic, abdominal, and arthroscopic.

A further important aspect is the ability to make the instrument disposable. The disposable element is preferably the instrument insert 16 such as illustrated in FIG. 15A. This disposable unit may be considered as comprising a disposable, mechanically drivable mechanism such as the coupler 300 interconnected to a disposable tool 18 through a disposable elongated tube such as the stem section 301, 302 of the instrument insert. This disposable implement is mounted so that the mechanically drivable mechanism may be connectable to and drivable from a drive mechanism. In the illustrated embodiment the drive mechanism may include the coupler 230 and the associated drive motors. The disposable elongate tube 301, 302 is inserted into an incision or orifice of a patient along a selected length of the disposable elongated tube.

The aforementioned disposable implement is purely mechanical and can be constructed relatively inexpensively, thus lending itself readily to being disposable. Another factor that lends itself to disposability is the simplicity of the instrument distal end tool (and wrist) construction. Prior tool constructions, whether graspers or other types, are relatively complex in that they usually have multiple pulleys at the wrist location for operation of different degrees-of-freedom there, making the structure quite intricate and relatively expensive to manufacture. On the other hand, in accordance with the present invention, no pulleys are required and the mechanism in the location of the wrist and tool is simple in construction and can be manufactured at far less expense, thus readily lending itself to disposability. One of the aspects of the invention that has enabled elimination of the pulleys, or the like, is the decoupling of tool action relative to wrist action by passing the tool actuation cables essentially through the center axis (604 in FIGS. 16A and 16B) of the wrist joint. This construction allows proper wrist action without any significant action being conveyed to the tool cables, and furthermore allows for a very simple and inexpensive construction at the distal end of the implement

Another aspect is the relative simplicity of the system, both in its construction and use. This provides an instrument system that is far less complex than prior robotic systems. Furthermore, by enabling a decoupling of the slave unit at the motor array, there is provided a readily portable and readily manually insertable slave unit that can be handled quite effectively by the surgeon or assistant when the slave unit is to be engaged through a patient incision or orifice. This enables the slave unit to be positioned through the incision or orifice so as to dispose the distal end at a target or operative site. A support is then preferably provided so as to hold a base of the slave unit fixed in position relative to the patient at least during a procedure that is to be carried out. This initial positioning of the slave unit with a predefined configuration immediately establishes an initial reference position for the instrument from which control occurs via a controller and user interface.

This portable nature of the slave unit comes about by virtue of providing a relatively simple surgical instrument insert in combination with an adaptor for the insert that is of relatively small configuration, particularly compared with prior large articulated robotic arm(s) structures. Because the slave unit is purely mechanical, and is decouplable from the drive unit, the slave unit can be readily positioned by the operator. Once in position, the unit is then secured to the support and the mechanical cables are coupled with the drive unit. This makes the slave unit both portable and easy to position in place for use.

Another advantage of the system is the ability to position the holder or adaptor for the instrument with its distal end at the operative site and maintained at the operative site even during instrument exchange. By way of example, and with reference to FIG. 2B, the instrument holder is represented by the guide tube 17 extending to the operative site OS. When instruments are to be exchanged, the distal end of the guide tube 17 essentially remains in place and the appropriate instruments are simply inserted and/or withdrawn depending on the particular procedure that is being carried out.

Accordingly, one of the advantages is the ease of exchanging instruments. In a particular operation procedure, there may be a multitude of instrument exchanges and the present system is readily adapted for quick and easy instrument exchange. Because the holder or adaptor is maintained in position, the surgeon does not have to be as careful each and every time that he reintroduces an instrument into the patient. In previous systems, the instrument is only supported through a cannula at the area of the incision and when an instrument exchange is to occur, these systems require removal of the entire assembly. This means that each time a new instrument is introduced, great care is required to reposition the distal end of the instrument so as to avoid internal tissue or organ damage. On the other hand, in accordance with the present invention, because the holder or adaptor is maintained in position at the operative site, even during instrument exchange, the surgeon does not have to be as careful as the insert simply slides through the rigid tube adaptor. This also essentially eliminates any chance of tissue or organ damage during this instrument exchange.

Having now described a limited number of embodiments of the present invention, it should be apparent to those skilled in the art that numerous other embodiments and modifications thereof are contemplated as falling within the scope of the present invention.

1. A medical system, comprising:
a rigid support affixed relative to a patient table;
a carriage assembly mounted to the rigid support;
a surgical instrument mounted to the carriage assembly, the surgical instrument carrying a distal tool configured for performing a medical procedure on a patient; and
a drive unit configured for pivoting the carriage assembly about the rigid support and for linearly moving the surgical instrument relative to the rigid support.

2. The medical system of claim 1, wherein the rigid support is a rigid post mounted to the patient table.

3. The medical system of claim 1, wherein the carriage assembly comprises at least one rail and a carriage slidably disposed on the at least one rail, the surgical instrument affixed to the carriage.

4. The medical system of claim 1, further comprising a yoke mounted to the rigid support, wherein the carriage assembly further comprises a pivot piece pivotably secured to the yoke.

5. The medical system of claim 4, further comprising a bracket mounted to the rigid support, and a pin removably mounting the yoke to the rigid support.

6. The medical system of claim 1, wherein the drive unit has a motor array.

7. The medical system of claim 1, wherein the drive unit is further configured for rotating at least a portion of the surgical instrument about its longitudinal axis.

8. The medical system of claim 7, further comprising a base piece mounted to the carriage, wherein the drive unit is configured for rotating the at least portion of the surgical instrument within the base piece.

9. The medical system of claim 1, wherein the drive unit is further configured actuating the distal tool.

10. The medical system of claim 9, wherein the surgical instrument comprises an adapter to which the drive unit is coupled and an instrument insert carrying the distal tool and removably disposed within the adapter.

11. The medical system of claim 10, wherein the drive unit is configured for actuating the distal tool through the adapter.

12. The medical system of claim 1, wherein the drive unit is coupled to the surgical instrument via external cabling.

13. The medical system of claim 1, further comprising a remote controller configured for directing the drive unit to control movements of the surgical instrument.

14. The medical system of claim 13, wherein the remote controller is coupled to the drive unit via external cabling.

15. The medical system of claim 13, wherein the remote controller has a user interface for receiving commands from a user.

16. The medical system of claim 15, wherein the commands are movements made at the user interface that correspond to movements of the surgical instrument.

17. A medical system, comprising:
a rigid support affixed relative to a patient table;
an adapter supported by the rigid support;
an instrument insert removably disposed within the adapter, the instrument insert carrying a distal tool configured for performing a medical procedure on patient; and
a drive unit configured for pivoting the adapter about the rigid support, linearly moving the adapter relative to the rigid support, and rotating the adapter about its longitudinal axis.

18. The medical system of claim 17, wherein the rigid support is a rigid post mounted to the patient table.

19. The medical system of claim 17, wherein the adapter has an elongated guide member that receives the instrument insert.

20. The medical system of claim 17, wherein the drive unit has a motor array.

21. The medical system of claim 17, wherein the drive unit is further configured actuating the distal tool.

22. The medical system of claim 17, wherein the drive unit is coupled to the surgical instrument via external cabling.

23. The medical system of claim 17, further comprising a remote controller configured for directing the drive unit to control movements of the surgical instrument.

24. The medical system of claim 23, wherein the remote controller has a user interface for receiving commands from a user.

25. The medical system of claim 24, wherein the commands are movements made at the user interface that correspond to movements of the surgical instrument.
